SEQUENCE LISTING (98 sequences)

SEQ ID NO: 1 - DNA, 39 bp, synthetic construct
  misc_feature 1..39: note = Synthetic c5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA
cgccgcggtctcaggcgctgagtctgagtttacctgcgt

SEQ ID NO: 2 - DNA, 2137 bp, Homo sapiens
  source 1..2137: mol_type = genomic DNA
ccctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagcggcctcggacccaggtcctgcgcccaggtcctcccctgccccccagcgacggagccggggccgggggcggcggcgccgggggcatgcgggtgagccgcggctgcagaggcctgagcgcctgatcgccgcggacccgagccgagcccacccccctccccagccccccaccctggccgcgggggcggcgcgctcgatctacgcgttcggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggccttggagtagataatattgtttccctcgtccgtctgtctcgatgcctgattcggacggccaatggtgcctcccccacccctccacgtgtccgtccacccttccatcagcgggtctcctcccagcggcctccggctcttgcccagcagctcaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaaccc

SEQ ID NO: 3 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 4 - DNA, 2305 bp, Homo sapiens
  source 1..2305: mol_type = genomic DNA
ttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 5 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 6 - DNA, 3018 bp, Homo sapiens
  source 1..3018: mol_type = genomic DNA
gcccggagagccgcatctattggcagctttgttattgatcagaaactgctcgccgccgacttggcttccagtctggctgcgggcaacccttgagttttcgcctctgtcctgtcccccgaactgacaggtgctcccagcaacttgctggggacttctcgccgctcccccgcgtccccaccccctcattcctccctcgccttcacccccacccccaccacttcgccacagctcaggatttgtttaaaccttgggaaactggttcaggtccaggttttgctttgatccttttcaaaaactggagacacagaagagggctctaggaaaaagttttggatgggattatgtggaaactaccctgcgattctctgctgccagagcaggctcggcgcttccaccccagtgcagccttcccctggcggtggtgaaagagactcgggagtcgctgcttccaaagtgcccgccgtgagtgagctctcaccccagtcagccaaatgagcctcttcgggcttctcctgctgacatctgccctggccggccagagacaggggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcgtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatgatataaaatattgttgctctgacaaaaatacatgtatttcattctcgtatggtgctagagttagattaatctgcattttaaaaaactgaattggaatagaattggtaagttgcaaagactttttgaaaataattaaattatcatatcttccattcctgttattggagatgaaaataaaaagcaacttatgaaagtagacattcagatccagccattactaacctattccttttttggggaaatctgagcctagctcagaaaaacataaagcaccttgaaaaagacttggcagcttcctgataaagcgtgctgtgctgtgcagtaggaacacatcctatttattgtgatgttgtggttttattatcttaaactctgttccatacacttgtataaatacatggatatttttatgtacagaagtatgtctcttaaccagttcacttattgtactctggcaatttaaaagaaaatcagtaaaatattttgcttgtaaaatgcttaatatcgtgcctaggttatgtggtgactatttgaatcaaaaatgtattgaatcatcaaataaaagaatgtggctattttggggagaaaattaaaaaaaaaaa

SEQ ID NO: 7 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 8 - DNA, 3997 bp, Homo sapiens
  source 1..3997: mol_type = genomic DNA
tctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaactaaaaa

SEQ ID NO: 9 - protein, 370 aa, Homo sapiens
MHRLIFVYTLICANFCSCRDTSATPQSASIKALRNANLRRDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 10 - DNA, 3979 bp, Homo sapiens
  source 1..3979: mol_type = genomic DNA
tctcaggggccgcggccggggctggagaacgctgctgctccgctcgcctgccccgctagattcggcgctgcccgccccctgcagcctgtgctgcagctgccggccaccggagggggcgaacaaacaaacgtcaacctgttgtttgtcccgtcaccatttatcagctcagcaccacaaggaagtgcggcacccacacgcgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgtgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaatatggtttccagagaaaaggccaatacctaagcattttttccatgagaagcactgcatacttacctatgtggactataataacctgtctccaaaaccatgccataataatataagtgctttagaaattaaatcattgtgttttttatgcattttgctgaggcatgcttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaattaggtgggatatttgataggatccatatctaataatggattcgaactctccaaactacaccaattaatttaatgtatcttgcttttgtgttcccgtctttttgaaatatagacatggatttataatggcattttatatttggcaggccatcatagattatttacaacctaaaagcttttgtgtatcaaaaaaatcacattttattaatgtaaatttctaatcgtatacttgctcactgttctgatttcctgtttctgaaccaagtaaaatcagtcctagaggctatggttcttaatctatggagcttgctttaagaagccagttgtcaattgtggtaacacaagtttggccctgctgtcctactgtttaatagaaaactgttttacattggttaatggtatttagagtaattttttctctctgcctcctttgtgtctgttttaaaggagactaactccaggagtaggaaatgattcatcatcctccaaagcaagaggcttaagagagaaacaccgaaattcagatagctcagggactgctaacagagaactacatttttcttattgccttgaaagttaaaaggaaagcagatttcttcagtgactttgtggtcctactaactacaaccagtttgggtgacagggctggtaaagtcccagtgttagatgagtgacctaaatatacttagatttctaagtatggtgctctcaggtccaagttcaactattcttaagcagtgcaattcttcccagttatttgagatgaaagatctctgcttattgaagatgtaccttctaaaactttcctaaaagtgtctgatgtttttactcaagaggggagtggtaaaattaaatactctattgttcaattctctaaaatcccagaacacaatcagaaatagctcaggcagacactaataattaagaacgctcttcctcttcataactgctttgcaagtttcctgtgaaaacatcagtttcctgtaccaaagtcaaaatgaacgttacatcactctaacctgaacagctcacaatgtagctgtaaatataaaaaatgagagtgttctacccagttttcaataaaccttccaggctgcaataaccagcaaggttttcagttaaagccctatctgcactttttatttattagctgaaatgtaagcaggcatattcactcacttttctttgcctttcctgagagttttattaaaacttctcccttggttacctgttatcttttgcacttctaacatgtagccaataaatctatttgatagccatcaaaggaataaaaagctggccgtacaaattacatttcaaaacaaaccctaataaatccacatttccgcatggctcattcacctggaataatgccttttattgaatatgttcttatagggcaaaacactttcataagtagagttttttatgttttttgtcatatcggtaacatgcagctttttcctctcatagcattttctatagcgaatgtaatatgcctcttatcttcatgaaaaataaatattgcttttgaacaaaactaaaaa

SEQ ID NO: 11 - protein, 364 aa, Homo sapiens
MHRLIFVYTLICANFCSCRDTSATPQSASIKALRNANLRRDDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPR

SEQ ID NO: 12 - DNA, 6574 bp, Homo sapiens
  source 1..6574: mol_type = genomic DNA
aagagcaaaaagcgaaggcgcaatctggacactgggagattcggagcgcagggagtttgagagaaacttttattttgaagagaccaaggttgagggggggcttatttcctgacagctatttacttagagcaaatgattagttttagaaggatggactataacattgaatcaattacaaaacgcggtttttgagcccattactgttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggatgacatcggcatagactcttcagacctggtggaagacagcttcctgtaactggcggattcgaggggttccttccacttctggggccacctctggatcccgttcagaaaaccactttattgcaatgcagaggttgagaggaggacttggttgatgtttaaagagaagttcccagccaagggcctcggggagcgttctaaatatgaatgaatgggatattttgaaatgaactttgtcagtgttgcctcttgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttttcttcatgcctgatgaaagctttggcgaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggcattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgtgtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtta

SEQ ID NO: 13 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 14 - DNA, 5718 bp, Homo sapiens
  source 1..5718: mol_type = genomic DNA
ctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcacaaaaa

SEQ ID NO: 15 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 16 - DNA, 3626 bp, Homo sapiens
  source 1..3626: mol_type = genomic DNA
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 17 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 18 - DNA, 4017 bp, Homo sapiens
  source 1..4017: mol_type = genomic DNA
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctctatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctggaattattttaggaccaggaagcagcacgctgtttattgaaagagtcacagaagaggatgaaggtgtctatcactgcaaagccaccaaccagaagggctctgtggaaagttcagcatacctcactgttcaaggaacctcggacaagtctaatctggagctgatcactctaacatgcacctgtgtggctgcgactctcttctggctcctattaaccctctttatccgaaaaatgaaaaggtcttcttctgaaataaagactgactacctatcaattataatggacccagatgaagttcctttggatgagcagtgtgagcggctcccttatgatgccagcaagtgggagtttgcccgggagagacttaaactgggcaaatcacttggaagaggggcttttggaaaagtggttcaagcatcagcatttggcattaagaaatcacctacgtgccggactgtggctgtgaaaatgctgaaagagggggccacggccagcgagtacaaagctctgatgactgagctaaaaatcttgacccacattggccaccatctgaacgtggttaacctgctgggagcctgcaccaagcaaggagggcctctgatggtgattgttgaatactgcaaatatggaaatctctccaactacctcaagagcaaacgtgacttattttttctcaacaaggatgcagcactacacatggagcctaagaaagaaaaaatggagccaggcctggaacaaggcaagaaaccaagactagatagcgtcaccagcagcgaaagctttgcgagctccggctttcaggaagataaaagtctgagtgatgttgaggaagaggaggattctgacggtttctacaaggagcccatcactatggaagatctgatttcttacagttttcaagtggccagaggcatggagttcctgtcttccagaaagtgcattcatcgggacctggcagcgagaaacattcttttatctgagaacaacgtggtgaagatttgtgattttggccttgcccgggatatttataagaaccccgattatgtgagaaaaggagatactcgacttcctctgaaatggatggctcctgaatctatctttgacaaaatctacagcaccaagagcgacgtgtggtcttacggagtattgctgtgggaaatcttctccttaggtgggtctccatacccaggagtacaaatggatgaggacttttgcagtcgcctgagggaaggcatgaggatgagagctcctgagtactctactcctgaaatctatcagatcatgctggactgctggcacagagacccaaaagaaaggccaagatttgcagaacttgtggaaaaactaggtgatttgcttcaagcaaatgtacaacaggatggtaaagactacatcccaatcaatgccatactgacaggaaatagtgggtttacatactcaactcctgccttctctgaggacttcttcaaggaaagtatttcagctccgaagtttaattcaggaagctctgatgatgtcagatatgtaaatgctttcaagttcatgagcctggaaagaatcaaaacctttgaagaacttttaccgaatgccacctccatgtttgatgactaccagggcgacagcagcactctgttggcctctcccatgctgaagcgcttcacctggactgacagcaaacccaaggcctcgctcaagattgacttgagagtaaccagtaaaagtaaggagtcggggctgtctgatgtcagcaggcccagtttctgccattccagctgtgggcacgtcagcgaaggcaagcgcaggttcacctacgaccacgctgagctggaaaggaaaatcgcgtgctgctccccgcccccagactacaactcggtggtcctgtactccaccccacccatctag

SEQ ID NO: 19 - protein, 1338 aa, Homo sapiens [sequence omitted]

SEQ ID NO: 20 - DNA, 5830 bp, Homo sapiens
  source 1..5830: mol_type = genomic DNA
actgagtcccgggaccccgggagagcggtcagtgtgtggtcgctgcgtttcctctgcctgcgccgggcatcacttgcgcgccgcagaaagtccgtctggcagcctggatatcctctcctaccggcacccgcagacgcccctgcagccgccggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgcaggatgcagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaaaaggaagcatccacaccccaactcccggacatcacatgagaggtctgctcagattttgaagtgttgttctttccaccagcaggaagtagccgcatttgattttcatttcgacaacagaaaaaggacctcggactgcagggagccagtcttctaggcatatcctggaagaggcttgtgacccaagaatgtgtctgtgtcttctcccagtgttgacctgatcctcttttttcattcatttaaaaagcattatcatgcccctgctgcgggtctcaccatgggtttagaacaaagagcttcaagcaatggccccatcctcaaagaagtagcagtacctggggagctgacacttctgtaaaactagaagataaaccaggcaacgtaagtgttcgaggtgttgaagatgggaaggatttgcagggctgagtctatccaagaggctttgtttaggacgtgggtcccaagccaagccttaagtgtggaattcggattgatagaaaggaagactaacgttaccttgctttggagagtactggagcctgcaaatgcattgtgtttgctctggtggaggtgggcatggggtctgttctgaaatgtaaagggttcagacggggtttctggttttagaaggttgcgtgttcttcgagttgggctaaagtagagttcgttgtgctgtttctgactcctaatgagagttccttccagaccgttagctgtctccttgccaagccccaggaagaaaatgatgcagctctggctccttgtctcccaggctgatcctttattcagaataccacaaagaaaggacattcagctcaaggctccctgccgtgttgaagagttctgactgcacaaaccagcttctggtttcttctggaatgaataccctcatatctgtcctgatgtgatatgtctgagactgaatgcgggaggttcaatgtgaagctgtgtgtggtgtcaaagtttcaggaaggattttacccttttgttcttccccctgtccccaacccactctcaccccgcaacccatcagtattttagttatttggcctctactccagtaaacctgattgggtttgttcactctctgaatgattattagccagacttcaaaattattttatagcccaaattataacatctattgtattatttagacttttaacatatagagctatttctactgatttttgcccttgttctgtcctttttttcaaaaaagaaaatgtgttttttgtttggtaccatagtgtgaaatgctgggaacaatgactataagacatgctatggcacatatatttatagtctgtttatgtagaaacaaatgtaatatattaaagccttatatataatgaactttgtactattcacattttgtatcagtattatgtagcataacaaaggtcataatgctttcagcaattgatgtcattttattaaagaacattgaaaaacttga

SEQ ID NO: 21 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 22 - protein, 16 aa, Homo sapiens
CNDEGLECVPTEESNI

SEQ ID NO: 23 - protein, 16 aa, Homo sapiens
CPDDGLECVPTGQHQV

SEQ ID NO: 24 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 25 - RNA, 42 nt, synthetic construct
  misc_feature 1..42: note = Synthetic C5 specific aptamer
  misc_difference 1: note = May be 2'OH-guanosine
  misc_difference 2: note = May be 2'OH-adenosine
  misc_difference 3: note = May be 2'-fluoro-2'-deoxycytidine
  misc_difference 4: note = May be 2'OH-guanosine
  misc_difference 5: note = May be 2'OH-adenosine
  misc_difference 6: note = May be 2'-fluoro-2'-deoxyuridine
  misc_difference 7: note = May be 2'OH-guanosine
  misc_difference 8: note = May be 2'-fluoro-2'-deoxycytidine
  misc_difference 9: note = May be 2'OH-guanosine
  misc_difference 10: note = May be 2'OH-guanosine
  misc_difference 11: note = May be 2'-fluoro-2'-deoxyuridine
  misc_difference 12: note = May be 2'-fluoro-2'-deoxycytidine
  misc_difference 13: note = May be 2'-fluoro-2'-deoxyuridine
  misc_difference 14: note = May be 2'-fluoro-2'-deoxycytidine
  misc_difference 15: note = May be 2'OH-adenosine
  misc_difference 16: note = May be 2'-fluoro-2'-deoxyuridine
  misc_difference 17: note = May be 2'OH-guanosine
  misc_difference 18: note = May be 2'-fluoro-2'-deoxycytidine
  misc_difference 19: note = May be 2'OH-guanosine
  misc_difference 20: note = May be 2'-fluoro-2'-deoxyuridine
  misc_difference 21: note = May be 2'-fluoro-2'-deoxycytidine
  misc_difference 22: note = May be 2'OH-guanosine
  misc_difference 23: note = May be 2'OH-adenosine
  misc_difference 24: note = May be 2'OH-guanosine
  misc_difference 25: note = May be 2'-fluoro-2'-deoxyuridine
  misc_difference 26: note = May be 2'OH-guanosine
  misc_difference 27: note = May be 2'-fluoro-2'-deoxyuridine
  misc_difference 28: note = May be 2'OH-guanosine
  misc_difference 29: note = May be 2'OH-adenosine
  misc_difference 30: note = May be 2'OH-guanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxycytidine
  misc_difference 37: note = May be 2'-fluoro-2'-deoxyuridine
  misc_difference 38: note = May be 2'-fluoro-2'-deoxyuridine
  misc_difference 39: note = May be 2'-fluoro-2'-deoxycytidine
  misc_difference 40: note = May be 2'OH-guanosine
  misc_difference 41: note = May be 2'-fluoro-2'-deoxyuridine
  misc_difference 42: note = May be 2'-fluoro-2'-deoxycytidine
  source 1..42: mol_type = other RNA
gacgatgcggtctcatgcgtcgagtgtgagtttaccttcgtc

SEQ ID NO: 26 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'OH-adenosine
  misc_difference 32: note = May be 2'-fluoro-2'-deoxycytidine
  misc_difference 33: note = May be 2'-fluoro-2'-deoxycytidine
  misc_difference 34: note = May be 2'-fluoro-2'-deoxyuridine
  misc_difference 35: note = May be 2'-O-Methyl-2'-deoxyguanosine
  misc_difference 36: note = May be 2'-fluoro-2'-deoxycytidine
  misc_difference 37: note = May be 2'-O-Methyl-2'-deoxyguanosine
  misc_feature 38: note = May be an inverted orientation T (3'-3'-linked)
  source 1..39: mol_type = other DNA

SEQ ID NO: 27 - RNA, 44 nt, synthetic construct
  misc_feature 1..44: note = Synthetic C5 specific aptamer
  misc_difference 1: note = May be 2'OH-adenosine
  misc_difference 2: note = May be 2'OH-guanosine
  misc_difference 3: note = May be 2'OH-guanosine
  misc_difference 4: note = May be 2'OH-adenosine
  misc_difference 5: note = May be 2'-fluoro-2'-deoxycytidine
  misc_difference 6: note = May be 2'OH-guanosine
  misc_difference 7: note = May be 2'OH-adenosine
  misc_difference 8: note = May be 2'-fluoro-2'-deoxyuridine
  misc_difference 9: note = May be 2'OH-guanosine
  misc_difference 10: note = May be 2'-fluoro-2'-deoxycytidine
  misc_difference 11: note = May be 2'OH-guanosine
  misc_difference 12: note = May be 2'OH-guanosine
  misc_difference 13: note = May be 2'-fluoro-2'-deoxyuridine
  misc_difference 14: note = May be 2'-fluoro-2'-deoxycytidine
  misc_difference 15: note = May be 2'-fluoro-2'-deoxyuridine
  misc_difference 16: note = May be 2'-fluoro-2'-deoxycytidine
  misc_difference 17: note = May be 2'OH-adenosine
  misc_difference 18: note = May be 2'-fluoro-2'-deoxyuridine
  misc_difference 19: note = May be 2'OH-guanosine
  misc_difference 20: note = May be 2'-fluoro-2'-deoxycytidine
  misc_difference 21: note = May be 2'OH-guanosine
  misc_difference 22: note = May be 2'-fluoro-2'-deoxyuridine
  misc_difference 23: note = May be 2'-fluoro-2'-deoxycytidine
  misc_difference 24: note = May be 2'OH-guanosine
  misc_difference 25: note = May be 2'OH-adenosine
  misc_difference 26: note = May be 2'OH-guanosine
  misc_difference 27: note = May be 2'-fluoro-2'-deoxyuridine
  misc_difference 28: note = May be 2'OH-guanosine
  misc_difference 29: note = May be 2'-fluoro-2'-deoxyuridine
  misc_difference 30: note = May be 2'OH-guanosine
  misc_difference 31: note = May be 2'OH-adenosine
  misc_difference 32: note = May be 2'OH-guanosine
  misc_difference 33: note = May be 2'-fluoro-2'-deoxyuridine
  misc_difference 34: note = May be 2'-fluoro-2'-deoxyuridine
  misc_difference 35: note = May be 2'-fluoro-2'-deoxyuridine
  misc_difference 36: note = May be 2'OH-adenosine
  misc_difference 37: note = May be 2'-fluoro-2'-deoxycytidine
  misc_difference 38: note = May be 2'-fluoro-2'-deoxycytidine
  misc_difference 39: note = May be 2'-fluoro-2'-deoxyuridine
  misc_difference 40: note = May be 2'-fluoro-2'-deoxyuridine
  misc_difference 41: note = May be 2'-fluoro-2'-deoxycytidine
  misc_difference 42: note = May be 2'OH-guanosine
  misc_difference 43: note = May be 2'-fluoro-2'-deoxyuridine
  misc_difference 44: note = May be 2'-fluoro-2'-deoxycytidine
  source 1..44: mol_type = other RNA
aggacgatgcggtctcatgcgtcgagtgtgagtttaccttcgtc

SEQ ID NO: 28 - RNA, 40 nt, synthetic construct
  misc_feature 1..40: note = Synthetic C5 specific aptamer
  misc_difference 1: note = May be 2'OH-adenosine
  misc_difference 2: note = May be 2'OH-guanosine
  misc_difference 3: note = May be 2'-fluoro-2'-deoxycytidine
  misc_difference 4: note = May be 2'-O-Methyl-2'-deoxyguanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'-fluoro-2'-deoxyuridine
  misc_difference 12: note = May be 2'-fluoro-2'-deoxycytidine
  misc_difference 13: note = May be 2'-fluoro-2'-deoxyuridine
  misc_difference 14: note = May be 2'-fluoro-2'-deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be 2'-fluoro-2'-deoxycytidine
  misc_difference 19: note = May be 2'OH-guanosine
  misc_difference 20: note = May be 2'-fluoro-2'-deoxycytidine
  misc_difference 21: note = May be 2'-fluoro-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'-fluoro-2'-deoxyuridine
  misc_difference 26: note = May be 2'-fluoro-2'-deoxycytidine
  misc_difference 27: note = May be 2'-fluoro-2'-deoxyuridine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxycytidine
  misc_difference 37: note = May be 2'-fluoro-2'-deoxyuridine
  misc_difference 38: note = May be 2'-O-Methyl-2'-deoxyguanosine
  misc_difference 39: note = May be 2'-fluoro-2'-deoxycytidine
  misc_difference 40: note = May be 2'-O-Methyl-2'-deoxyguanosine
  source 1..40: mol_type = other RNA
agcgccgcggtctcaggcgctgagtctgagtttacctgcg

SEQ ID NO: 29 - RNA, 46 nt, synthetic construct
  misc_feature 1..46: note = Synthetic C5 specific aptamer
  misc_difference 1: note = May be 2'OH-guanosine
  misc_difference 2: note = May be 2'OH-guanosine
  misc_difference 3: note = May be 2'-fluoro-2'-deoxycytidine
  misc_difference 4: note = May be 2'OH-guanosine
  misc_difference 5: note = May be 2'OH-adenosine
  misc_difference 6: note = May be 2'-fluoro-2'-deoxyuridine
  misc_difference 7: note = May be 2'-fluoro-2'-deoxyuridine
  misc_difference 8: note = May be 2'OH-adenosine
  misc_difference 9: note = May be 2'-fluoro-2'-deoxycytidine
  misc_difference 10: note = May be 2'-fluoro-2'-deoxyuridine
  misc_difference 11: note = May be 2'OH-guanosine
  misc_difference 12: note = May be 2'OH-guanosine
  misc_difference 13: note = May be 2'OH-guanosine
  misc_difference 14: note = May be 2'OH-adenosine
  misc_difference 15: note = May be 2'-fluoro-2'-deoxycytidine
  misc_difference 16: note = May be 2'OH-guanosine
  misc_difference 17: note = May be 2'OH-guanosine
  misc_difference 18: note = May be 2'OH-adenosine
  misc_difference 19: note = May be 2'-fluoro-2'-deoxycytidine
  misc_difference 20: note = May be 2'-fluoro-2'-deoxyuridine
  misc_difference 21: note = May be 2'-fluoro-2'-deoxycytidine
  misc_difference 22: note = May be 2'OH-guanosine
  misc_difference 23: note = May be 2'-fluoro-2'-deoxycytidine
  misc_difference 24: note = May be 2'OH-guanosine
  misc_difference 25: note = May be 2'OH-adenosine
  misc_difference 26: note = May be 2'-fluoro-2'-deoxyuridine
  misc_difference 27: note = May be 2'OH-guanosine
  misc_difference 28: note = May be 2'-fluoro-2'-deoxyuridine
  misc_difference 29: note = May be 2'OH-guanosine
  misc_difference 30: note = May be 2'OH-adenosine
  misc_difference 31: note = May be 2'OH-guanosine
  misc_difference 32: note = May be 2'-fluoro-2'-deoxycytidine
  misc_difference 33: note = May be 2'-fluoro-2'-deoxycytidine
  misc_difference 34: note = May be 2'-fluoro-2'-deoxycytidine
  misc_difference 35: note = May be 2'OH-adenosine
  misc_difference 36: note = May be 2'OH-guanosine
  misc_difference 37: note = May be 2'OH-adenosine
  misc_difference 38: note = May be 2'-fluoro-2'-deoxycytidine
  misc_difference 39: note = May be 2'OH-guanosine
  misc_difference 40: note = May be 2'OH-adenosine
  misc_difference 41: note = May be 2'-fluoro-2'-deoxycytidine
  misc_difference 42: note = May be 2'-fluoro-2'-deoxyuridine
  misc_difference 43: note = May be 2'-fluoro-2'-deoxycytidine
  misc_difference 44: note = May be 2'OH-guanosine
  misc_difference 45: note = May be 2'-fluoro-2'-deoxycytidine
  misc_difference 46: note = May be 2'-fluoro-2'-deoxycytidine
  source 1..46: mol_type = other RNA
ggcgattactgggacggactcgcgatgtgagcccagacgactcgcc

SEQ ID NO: 30 - RNA, 40 nt, synthetic construct
  misc_feature 1..40: note = Synthetic C5 specific aptamer
  misc_difference 1: note = May be 2'OH-guanosine
  misc_difference 2: note = May be 2'OH-guanosine
  misc_difference 3: note = May be 2'-fluoro-2'-deoxycytidine
  misc_difference 4: note = May be 2'-fluoro-2'-deoxyuridine
  misc_difference 5: note = May be 2'-fluoro-2'-deoxyuridine
  misc_difference 6: note = May be 2'-fluoro-2'-deoxycytidine
  misc_difference 7: note = May be 2'-fluoro-2'-deoxyuridine
  misc_difference 8: note = May be 2'OH-guanosine
  misc_difference 9: note = May be 2'OH-adenosine
  misc_difference 10: note = May be 2'OH-adenosine
  misc_difference 11: note = May be 2'OH-guanosine
  misc_difference 12: note = May be 2'OH-adenosine
  misc_difference 13: note = May be 2'-fluoro-2'-deoxyuridine
  misc_difference 14: note = May be 2'-fluoro-2'-deoxyuridine
  misc_difference 15: note = May be 2'OH-adenosine
  misc_difference 16: note = May be 2'-fluoro-2'-deoxyuridine
  misc_difference 17: note = May be 2'-fluoro-2'-deoxyuridine
  misc_difference 18: note = May be 2'-fluoro-2'-deoxyuridine
  misc_difference 19: note = May be 2'-fluoro-2'-deoxycytidine
  misc_difference 20: note = May be 2'OH-guanosine
  misc_difference 21: note = May be 2'-fluoro-2'-deoxycytidine
  misc_difference 22: note = May be 2'OH-guanosine
  misc_difference 23: note = May be 2'OH-adenosine
  misc_difference 24: note = May be 2'-fluoro-2'-deoxyuridine
  misc_difference 25: note = May be 2'OH-guanosine
  misc_difference 26: note = May be 2'-fluoro-2'-deoxyuridine
  misc_difference 27: note = May be 2'OH-guanosine
  misc_difference 28: note = May be 2'OH-adenosine
  misc_difference 29: note = May be 2'OH-adenosine
  misc_difference 30: note = May be 2'-fluoro-2'-deoxycytidine
  misc_difference 31: note = May be 2'-fluoro-2'-deoxyuridine
  misc_difference 32: note = May be 2'-fluoro-2'-deoxycytidine
  misc_difference 33: note = May be 2'-fluoro-2'-deoxycytidine
  misc_difference 34: note = May be 2'OH-adenosine
  misc_difference 35: note = May be 2'OH-guanosine
  misc_difference 36: note = May be 2'OH-adenosine
  misc_difference 37: note = May be 2'-fluoro-2'-deoxycytidine
  misc_difference 38: note = May be 2'-fluoro-2'-deoxycytidine
  misc_difference 39: note = May be 2'-fluoro-2'-deoxycytidine
  misc_difference 40: note = May be 2'-fluoro-2'-deoxycytidine
  source 1..40: mol_type = other RNA
ggcttctgaagattatttcgcgatgtgaactccagacccc

SEQ ID NO: 31 - RNA, 40 nt, synthetic construct
  misc_feature 1..40: note = Synthetic C5 specific aptamer
  misc_difference 1: note = May be 2'OH-guanosine
  misc_difference 2: note = May be 2'OH-guanosine
  misc_difference 3: note = May be 2'-fluoro-2'-deoxycytidine
  misc_difference 4: note = May be 2'OH-guanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'OH-guanosine
  misc_difference 10: note = May be 2'OH-guanosine
  misc_difference 11: note = May be 2'-fluoro-2'-deoxyuridine
  misc_difference 12: note = May be 2'-fluoro-2'-deoxycytidine
  misc_difference 13: note = May be 2'-fluoro-2'-deoxyuridine
  misc_difference 14: note = May be 2'-fluoro-2'-deoxycytidine
  misc_difference 15: note = May be 2'OH-adenosine
  misc_difference 16: note = May be 2'OH-guanosine
  misc_difference 17: note = May be 2'OH-guanosine
  misc_difference 18: note = May be 2'-fluoro-2'-deoxycytidine
  misc_difference 19: note = May be 2'OH-guanosine
  misc_difference 20: note = May be 2'-fluoro-2'-deoxycytidine
  misc_difference 21: note = May be 2'-fluoro-2'-deoxyuridine
  misc_difference 22: note = May be 2'OH-guanosine
  misc_difference 23: note = May be 2'OH-adenosine
  misc_difference 24: note = May be 2'OH-guanosine
  misc_difference 25: note = May be 2'-fluoro-2'-deoxyuridine
  misc_difference 26: note = May be 2'-fluoro-2'-deoxycytidine
  misc_difference 27: note = May be 2'-fluoro-2'-deoxyuridine
  misc_difference 28: note = May be 2'OH-guanosine
  misc_difference 29: note = May be 2'OH-adenosine
  misc_difference 30: note = May be 2'OH-guanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxycytidine
  misc_difference 37: note = May be 2'-fluoro-2'-deoxyuridine
  misc_difference 38: note = May be 2'OH-guanosine
  misc_difference 39: note = May be 2'-fluoro-2'-deoxycytidine
  misc_difference 40: note = May be 2'OH-guanosine
  source 1..40: mol_type = other RNA
ggcgccgcggtctcaggcgctgagtctgagtttacctgcg

SEQ ID NO: 32 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be deoxycytidine
  misc_difference 34: note = May be deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 33 - DNA, 39 bp, synthetic construct; identical to SEQ ID NO: 1
  misc_feature 1..39: note = Synthetic C5 specific aptamer
  misc_difference 1: note = May be 2'-O-Methyl-2'-deoxycytidine
  misc_difference 2: note = May be 2'-O-Methyl-2'-deoxyguanosine
  misc_difference 3: note = May be 2'-O-Methyl-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be deoxycytidine
  misc_difference 34: note = May be deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-O-Methyl-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 34 - DNA, 38 bp, synthetic construct
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'OH-cytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA
cgccgcggtctcaggcgctgagtctgagtttacctgcg

SEQ ID NO: 35 - DNA, 38 bp, synthetic construct; identical to SEQ ID NO: 34
  misc_feature 1..38: note = Synthetic C5 specific aptamer
  misc_difference 1: note = May be deoxycytidine
  misc_difference 2: note = May be 2'-O-Methyl-2'-deoxyguanosine
  misc_difference 3: note = May be deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be deoxycytidine
  misc_difference 38: note = May be 2'-O-Methyl-2'-deoxyguanosine
  source 1..38: mol_type = other DNA

SEQ ID NO: 36 - DNA, 38 bp, synthetic construct; identical to SEQ ID NO: 34
  misc_feature 1..38: note = Synthetic C5 specific aptamer
  misc_difference 1: note = May be deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 37 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 38 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be deoxycytidine
  misc_difference 38: note = May be 2'-O-Methyl-2'-deoxyguanosine
  source 1..38: mol_type = other DNA

SEQ ID NO: 39 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-O-Methyl-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 40 - DNA, 37 bp, synthetic construct
  misc_feature 1..37: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxyuridine
  misc_difference 35: note = May be 2'-O-Methyl-2'-deoxyguanosine
  misc_difference 36: note = May be 2'-O-Methyl-2'-deoxycytidine
  misc_difference 37: note = May be 2'-O-Methyl-2'-deoxyguanosine
  source 1..37: mol_type = other DNA
cgccgcggtctcaggcgctgagtctgagtttactgcg

SEQ ID NO: 41 - DNA, 38 bp, synthetic construct; identical to SEQ ID NO: 34
  misc_feature 1..38: note = Synthetic C5 specific aptamer
  misc_difference 1: note = May be 2'-O-Methyl-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 42 - DNA, 37 bp, synthetic construct; identical to SEQ ID NO: 40
  misc_feature 1..37: note = Synthetic C5 specific aptamer
  misc_difference 1: note = May be 2'-O-Methyl-2'-deoxycytidine
  misc_difference 2: note = May be 2'-O-Methyl-2'-deoxyguanosine
  misc_difference 3: note = May be 2'-O-Methyl-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxyuridine
  misc_difference 35: note = May be 2'-O-Methyl-2'-deoxyguanosine
  misc_difference 36: note = May be 2'-O-Methyl-2'-deoxycytidine
  misc_difference 37: note = May be 2'-O-Methyl-2'-deoxyguanosine
  source 1..37: mol_type = other DNA

SEQ ID NO: 43 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'OH-cytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 44 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 45 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be deoxycytidine
  misc_difference 5: note = May be 2'OH-guanosine
  misc_difference 6: note = May be deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 46 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 47 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be deoxycytidine
  misc_difference 19: note = May be 2'OH-thymidine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 48 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be deoxycytidine
  misc_difference 19: note = May be 2'OH-thymidine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 49 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'OH-thymidine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 50 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'OH-thymidine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 51 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'OH-thymidine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 52 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'OH-thymidine
  misc_difference 30: note = May be 2'OH-thymidine
  misc_difference 31: note = May be 2'OH-thymidine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 53 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'OH-thymidine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 54 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-O-Methyl-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be deoxycytidine
  misc_difference 34: note = May be deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 55 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-O-Methyl-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 56 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-O-Methyl-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 57 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-O-Methyl-2'-deoxycytidine
  misc_difference 5: note = May be 2'OH-guanosine
  misc_difference 6: note = May be 2'-O-Methyl-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 58 - DNA, 37 bp, synthetic construct
  misc_feature 1..37: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'OH-guanosine
  misc_difference 5: note = May be 2'-fluoro-2'-deoxycytidine
  misc_difference 6: note = May be 2'-O-Methyl-2'-deoxyguanosine
  misc_difference 7: note = May be 2'-O-Methyl-2'-deoxyguanosine
  misc_difference 8: note = May be 2'-fluoro-2'-deoxyuridine
  misc_difference 9: note = May be deoxycytidine
  misc_difference 10: note = May be 2'-fluoro-2'-deoxyuridine
  misc_difference 11: note = May be deoxycytidine
  misc_difference 12: note = May be 2'-O-Methyl-2'-deoxyadenosine
  misc_difference 13: note = May be 2'-O-Methyl-2'-deoxyguanosine
  misc_difference 14: note = May be 2'-O-Methyl-2'-deoxyguanosine
  misc_difference 15: note = May be deoxycytidine
  misc_difference 16: note = May be 2'OH-guanosine
  misc_difference 17: note = May be 2'-O-Methyl-2'-deoxycytidine
  misc_difference 18: note = May be 2'-fluoro-2'-deoxyuridine
  misc_difference 19: note = May be 2'-O-Methyl-2'-deoxyguanosine
  misc_difference 20: note = May be 2'-O-Methyl-2'-deoxyadenosine
  misc_difference 21: note = May be 2'-O-Methyl-2'-deoxyguanosine
  misc_difference 22: note = May be 2'-fluoro-2'-deoxyuridine
  misc_difference 23: note = May be deoxycytidine
  misc_difference 24: note = May be 2'-fluoro-2'-deoxyuridine
  misc_difference 25: note = May be 2'-O-Methyl-2'-deoxyguanosine
  misc_difference 26: note = May be 2'-O-Methyl-2'-deoxyadenosine
  misc_difference 27: note = May be 2'-O-Methyl-2'-deoxyguanosine
  misc_difference 28: note = May be 2'-fluoro-2'-deoxyuridine
  misc_difference 29: note = May be 2'-fluoro-2'-deoxyuridine
  misc_difference 30: note = May be 2'-fluoro-2'-deoxyuridine
  misc_difference 31: note = May be 2'OH-adenosine
  misc_difference 32: note = May be 2'-fluoro-2'-deoxycytidine
  misc_difference 33: note = May be 2'-fluoro-2'-deoxycytidine
  misc_difference 34: note = May be 2'-fluoro-2'-deoxyuridine
  misc_difference 35: note = May be 2'-O-Methyl-2'-deoxyguanosine
  misc_difference 36: note = May be 2'-fluoro-2'-deoxycytidine
  misc_difference 37: note = May be 2'-O-Methyl-2'-deoxyguanosine
  source 1..37: mol_type = other DNA
cgcgcggtctcaggcgctgagtctgagtttacctgcg

SEQ ID NO: 59 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'-fluoro-2'-deoxyuridine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-O-Methyl-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 60 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'-fluoro-2'-deoxyuridine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-O-Methyl-2'-deoxycytidine
  misc_difference 19: note = May be 2'-O-Methyl-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 61 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-O-Methyl-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 62 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-O-Methyl-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 63 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-O-Methyl-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 64 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-O-Methyl-2'-deoxyuridine
  misc_difference 30: note = May be 2'-O-Methyl-2'-deoxyuridine
  misc_difference 31: note = May be 2'-O-Methyl-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 65 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-O-Methyl-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 66 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be deoxyguanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 67 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be deoxyguanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'OH-cytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 68 - DNA, 38 bp, synthetic construct; identical to SEQ ID NO: 34
  misc_feature 1..38: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be deoxyadenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  source 1..38: mol_type = other DNA

SEQ ID NO: 69 - DNA, 40 bp, synthetic construct
  misc_feature 1..40: note = Synthetic C5 specific aptamer
  misc_difference 1: note = May be 2'-O-Methyl-2'-deoxyguanosine
  misc_difference 2: note = May be 2'-fluoro-2'-deoxycytidine
  misc_difference 3: note = May be 2'-O-Methyl-2'-deoxyguanosine
  misc_difference 4: note = May be 2'-fluoro-2'-deoxyuridine
  misc_difference 5: note = May be 2'-fluoro-2'-deoxycytidine
  misc_difference 6: note = May be 2'OH-guanosine
  misc_difference 7: note = May be 2'-fluoro-2'-deoxycytidine
  misc_difference 8: note = May be 2'-O-Methyl-2'-deoxyguanosine
  misc_difference 9: note = May be 2'-O-Methyl-2'-deoxyguanosine
  misc_difference 10: note = May be 2'-fluoro-2'-deoxyuridine
  misc_difference 11: note = May be deoxycytidine
  misc_difference 12: note = May be 2'OH-thymidine
  misc_difference 13: note = May be deoxycytidine
  misc_difference 14: note = May be 2'-O-Methyl-2'-deoxyadenosine
  misc_difference 15: note = May be 2'-O-Methyl-2'-deoxyguanosine
  misc_difference 16: note = May be 2'-O-Methyl-2'-deoxyguanosine
  misc_difference 17: note = May be deoxycytidine
  misc_difference 18: note = May be 2'OH-guanosine
  misc_difference 19: note = May be 2'-fluoro-2'-deoxycytidine
  misc_difference 20: note = May be 2'-fluoro-2'-deoxyuridine
  misc_difference 21: note = May be 2'-O-Methyl-2'-deoxyguanosine
  misc_difference 22: note = May be 2'-O-Methyl-2'-deoxyadenosine
  misc_difference 23: note = May be 2'-O-Methyl-2'-deoxyguanosine
  misc_difference 24: note = May be 2'OH-thymidine
  misc_difference 25: note = May be deoxycytidine
  misc_difference 26: note = May be 2'OH-thymidine
  misc_difference 27: note = May be 2'-O-Methyl-2'-deoxyguanosine
  misc_difference 28: note = May be 2'-O-Methyl-2'-deoxyadenosine
  misc_difference 29: note = May be 2'-O-Methyl-2'-deoxyguanosine
  misc_difference 30: note = May be 2'-fluoro-2'-deoxyuridine
  misc_difference 31: note = May be 2'-fluoro-2'-deoxyuridine
  misc_difference 32: note = May be 2'-fluoro-2'-deoxyuridine
  misc_difference 33: note = May be 2'OH-adenosine
  misc_difference 34: note = May be 2'-fluoro-2'-deoxycytidine
  misc_difference 35: note = May be 2'-fluoro-2'-deoxycytidine
  misc_difference 36: note = May be 2'-fluoro-2'-deoxyuridine
  misc_difference 37: note = May be 2'-O-Methyl-2'-deoxyadenosine
  misc_difference 38: note = May be 2'-fluoro-2'-deoxycytidine
  misc_difference 39: note = May be 2'-O-Methyl-2'-deoxyguanosine
  misc_difference 40: note = May be 2'-O-Methyl-2'-deoxycytidine
  source 1..40: mol_type = other DNA
gcgtcgcggtctcaggcgctgagtctgagtttacctacgc

SEQ ID NO: 70 - DNA, 38 bp, synthetic construct
  misc_feature 1..38: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-O-Methyl-2'-deoxyguanosine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be deoxycytidine
  misc_difference 11: note = May be 2'OH-thymidine
  misc_difference 12: note = May be deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'OH-thymidine
  misc_difference 24: note = May be deoxycytidine
  misc_difference 25: note = May be 2'OH-thymidine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxycytidine
  misc_difference 37: note = May be 2'-O-Methyl-2'-deoxycytidine
  misc_difference 38: note = May be 2'-O-Methyl-2'-deoxycytidine
  source 1..38: mol_type = other DNA
gggcgcggtctcaggcgctgagtctgagtttacctccc

SEQ ID NO: 71 - DNA, 39 bp, synthetic construct
  misc_feature 1..39: note = Synthetic C5 specific aptamer
  misc_difference 1: note = May be 2'-O-Methyl-2'-deoxyguanosine
  misc_difference 2: note = May be 2'-fluoro-2'-deoxycytidine
  misc_difference 3: note = May be 2'-O-Methyl-2'-deoxyguanosine
  misc_difference 4: note = May be 2'-fluoro-2'-deoxycytidine
  misc_difference 5: note = May be 2'-fluoro-2'-deoxycytidine
  misc_difference 6: note = May be 2'OH-guanosine
  misc_difference 7: note = May be 2'-fluoro-2'-deoxycytidine
  misc_difference 8: note = May be 2'-O-Methyl-2'-deoxyguanosine
  misc_difference 9: note = May be 2'-O-Methyl-2'-deoxyguanosine
  misc_difference 10: note = May be 2'-fluoro-2'-deoxyuridine
  misc_difference 11: note = May be deoxycytidine
  misc_difference 12: note = May be 2'OH-thymidine
  misc_difference 13: note = May be deoxycytidine
  misc_difference 14: note = May be 2'-O-Methyl-2'-deoxyadenosine
  misc_difference 15: note = May be 2'-O-Methyl-2'-deoxyguanosine
  misc_difference 16: note = May be 2'-O-Methyl-2'-deoxyguanosine
  misc_difference 17: note = May be deoxycytidine
  misc_difference 18: note = May be 2'OH-guanosine
  misc_difference 19: note = May be 2'-fluoro-2'-deoxycytidine
  misc_difference 20: note = May be 2'-fluoro-2'-deoxyuridine
  misc_difference 21: note = May be 2'-O-Methyl-2'-deoxyguanosine
  misc_difference 22: note = May be 2'-O-Methyl-2'-deoxyadenosine
  misc_difference 23: note = May be 2'-O-Methyl-2'-deoxyguanosine
  misc_difference 24: note = May be 2'OH-thymidine
  misc_difference 25: note = May be deoxycytidine
  misc_difference 26: note = May be 2'OH-thymidine
  misc_difference 27: note = May be 2'-O-Methyl-2'-deoxyguanosine
  misc_difference 28: note = May be 2'-O-Methyl-2'-deoxyadenosine
  misc_difference 29: note = May be 2'-O-Methyl-2'-deoxyguanosine
  misc_difference 30: note = May be 2'-fluoro-2'-deoxyuridine
  misc_difference 31: note = May be 2'-fluoro-2'-deoxyuridine
  misc_difference 32: note = May be 2'-fluoro-2'-deoxyuridine
  misc_difference 33: note = May be 2'OH-adenosine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_difference 39: note = May be 2'-O-Methyl-2'-deoxycytidine
  source 1..39: mol_type = other DNA
gcgccgcggtctcaggcgctgagtctgagtttactgcgc

SEQ ID NO: 72 - DNA, 43 bp, synthetic construct
  misc_feature 1..43: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-O-Methyl-2'-deoxyadenosine
  misc_difference 4: note = May be 2'-fluoro-2'-deoxycytidine
  misc_difference 5: note = May be 2'-O-Methyl-2'-deoxyguanosine
  misc_difference 6: note = May be 2'-fluoro-2'-deoxycytidine
  misc_difference 7: note = May be 2'-fluoro-2'-deoxycytidine
  misc_difference 8: note = May be 2'OH-guanosine
  misc_difference 9: note = May be 2'-fluoro-2'-deoxycytidine
  misc_difference 10: note = May be 2'-O-Methyl-2'-deoxyguanosine
  misc_difference 11: note = May be 2'-O-Methyl-2'-deoxyguanosine
  misc_difference 12: note = May be 2'-fluoro-2'-deoxyuridine
  misc_difference 13: note = May be 2'-fluoro-2'-deoxycytidine
  misc_difference 14: note = May be 2'-fluoro-2'-deoxyuridine
  misc_difference 15: note = May be 2'-fluoro-2'-deoxycytidine
  misc_difference 16: note = May be 2'-O-Methyl-2'-deoxyadenosine
  misc_difference 17: note = May be 2'-O-Methyl-2'-deoxyguanosine
  misc_difference 18: note = May be 2'-O-Methyl-2'-deoxyguanosine
  misc_difference 19: note = May be 2'-fluoro-2'-deoxycytidine
  misc_difference 20: note = May be 2'OH-guanosine
  misc_difference 21: note = May be 2'-fluoro-2'-deoxycytidine
  misc_difference 22: note = May be 2'-fluoro-2'-deoxyuridine
  misc_difference 23: note = May be 2'-O-Methyl-2'-deoxyguanosine
  misc_difference 24: note = May be 2'-O-Methyl-2'-deoxyadenosine
  misc_difference 25: note = May be 2'-O-Methyl-2'-deoxyguanosine
  misc_difference 26: note = May be 2'-fluoro-2'-deoxyuridine
  misc_difference 27: note = May be 2'-fluoro-2'-deoxycytidine
  misc_difference 28: note = May be 2'-fluoro-2'-deoxyuridine
  misc_difference 29: note = May be 2'-O-Methyl-2'-deoxyguanosine
  misc_difference 30: note = May be 2'-O-Methyl-2'-deoxyguanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxyuridine
  misc_difference 37: note = May be 2'-O-Methyl-2'-deoxyguanosine
  misc_difference 38: note = May be 2'-fluoro-2'-deoxycytidine
  misc_difference 39: note = May be 2'-O-Methyl-2'-deoxyguanosine
  misc_difference 40: note = May be 2'-fluoro-2'-deoxyuridine
  misc_difference 41: note = May be 2'-fluoro-2'-deoxycytidine
  misc_difference 42: note = May be 2'-fluoro-2'-deoxyuridine
  misc_feature 43: note = May be an inverted orientation T (3'-3'-linked)
  source 1..43: mol_type = other DNA
ggacgccgcggtctcaggcgctgagtctggtttactgcgtctt

SEQ ID NO: 73 - DNA, 42 bp, synthetic construct
  misc_feature 1..42: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-fluoro-2'-deoxycytidine
  misc_difference 4: note = May be 2'-O-Methyl-2'-deoxyguanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'-fluoro-2'-deoxyuridine
  misc_difference 12: note = May be deoxycytidine
  misc_difference 13: note = May be 2'OH-thymidine
  misc_difference 14: note = May be deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be deoxycytidine
  misc_difference 19: note = May be 2'OH-guanosine
  misc_difference 20: note = May be 2'-O-Methyl-2'-deoxycytidine
  misc_difference 21: note = May be 2'-O-Methyl-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'OH-thymidine
  misc_difference 26: note = May be deoxycytidine
  misc_difference 27: note = May be 2'OH-thymidine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'OH-thymidine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be deoxycytidine
  misc_difference 36: note = May be deoxycytidine
  misc_difference 37: note = May be 2'OH-thymidine
  misc_difference 38: note = May be 2'-O-Methyl-2'-deoxyguanosine
  misc_difference 39: note = May be 2'-fluoro-2'-deoxycytidine
  misc_difference 40: note = May be 2'-O-Methyl-2'-deoxyguanosine
  misc_difference 41: note = May be 2'-O-Methyl-2'-deoxycytidine
  misc_difference 42: note = May be 2'-O-Methyl-2'-deoxycytidine
  source 1..42: mol_type = other DNA
ggcgccgcggtctcaggcgctgagtctgagtttacctgcgcc

SEQ ID NO: 74 - DNA, 40 bp, synthetic construct
  misc_feature 1..40: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-O-Methyl-2'-deoxycytidine
  misc_difference 4: note = May be 2'-O-Methyl-2'-deoxyguanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'-fluoro-2'-deoxyuridine
  misc_difference 12: note = May be deoxycytidine
  misc_difference 13: note = May be 2'OH-thymidine
  misc_difference 14: note = May be deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be deoxycytidine
  misc_difference 19: note = May be 2'OH-guanosine
  misc_difference 20: note = May be 2'-O-Methyl-2'-deoxycytidine
  misc_difference 21: note = May be 2'-O-Methyl-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'OH-thymidine
  misc_difference 26: note = May be deoxycytidine
  misc_difference 27: note = May be 2'OH-thymidine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxythymidine
  misc_difference 31: note = May be 2'OH-thymidine
  misc_difference 32: note = May be 2'OH-adenosine
  misc_difference 33: note = May be deoxycytidine
  misc_difference 34: note = May be deoxycytidine
  misc_difference 35: note = May be 2'OH-thymidine
  misc_difference 36: note = May be 2'-O-Methyl-2'-deoxyguanosine
  misc_difference 37: note = May be deoxycytidine
  misc_difference 38: note = May be 2'-O-Methyl-2'-deoxyguanosine
  misc_difference 39: note = May be 2'-O-Methyl-2'-deoxycytidine
  misc_difference 40: note = May be 2'-O-Methyl-2'-deoxycytidine
  source 1..40: mol_type = other DNA
ggcgccgcggtctcaggcgctgagtctgattacctgcgcc

SEQ ID NO: 75 - DNA, 42 bp, synthetic construct; identical to SEQ ID NO: 73
  misc_feature 1..42: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-O-Methyl-2'-deoxycytidine
  misc_difference 4: note = May be 2'-O-Methyl-2'-deoxyguanosine
  misc_difference 5: note = May be deoxycytidine
  misc_difference 6: note = May be deoxycytidine
  misc_difference 7: note = May be 2'OH-guanosine
  misc_difference 8: note = May be deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'OH-thymidine
  misc_difference 12: note = May be deoxycytidine
  misc_difference 13: note = May be 2'OH-thymidine
  misc_difference 14: note = May be deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be deoxycytidine
  misc_difference 19: note = May be 2'OH-guanosine
  misc_difference 20: note = May be 2'-O-Methyl-2'-deoxycytidine
  misc_difference 21: note = May be 2'-O-Methyl-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'OH-thymidine
  misc_difference 26: note = May be deoxycytidine
  misc_difference 27: note = May be 2'OH-thymidine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'OH-thymidine
  misc_difference 32: note = May be 2'OH-thymidine
  misc_difference 33: note = May be 2'OH-thymidine
  misc_difference 34: note = May be 2'-O-Methyl-2'-deoxyadenosine
  misc_difference 35: note = May be deoxycytidine
  misc_difference 36: note = May be deoxycytidine
  misc_difference 37: note = May be 2'OH-thymidine
  misc_difference 38: note = May be 2'-O-Methyl-2'-deoxyguanosine
  misc_difference 39: note = May be deoxycytidine
  misc_difference 40: note = May be 2'-O-Methyl-2'-deoxyguanosine
  misc_difference 41: note = May be 2'-O-Methyl-2'-deoxycytidine
  misc_difference 42: note = May be 2'-O-Methyl-2'-deoxycytidine
  source 1..42: mol_type = other DNA

SEQ ID NO: 76 - DNA, 42 bp, synthetic construct; identical to SEQ ID NO: 73
  misc_feature 1..42: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-O-Methyl-2'-deoxycytidine
  misc_difference 4: note = May be 2'-O-Methyl-2'-deoxyguanosine
  misc_difference 5: note = May be deoxycytidine
  misc_difference 6: note = May be deoxycytidine
  misc_difference 7: note = May be 2'OH-guanosine
  misc_difference 8: note = May be deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'OH-thymidine
  misc_difference 12: note = May be deoxycytidine
  misc_difference 13: note = May be 2'-O-Methyl-2'-deoxyuridine
  misc_difference 14: note = May be 2'-O-Methyl-2'-deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be deoxycytidine
  misc_difference 19: note = May be 2'OH-guanosine
  misc_difference 20: note = May be 2'-O-Methyl-2'-deoxycytidine
  misc_difference 21: note = May be 2'-O-Methyl-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'-O-Methyl-2'-deoxyuridine
  misc_difference 26: note = May be 2'-O-Methyl-2'-deoxycytidine
  misc_difference 27: note = May be 2'-O-Methyl-2'-deoxyuridine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'OH-thymidine
  misc_difference 32: note = May be 2'OH-thymidine
  misc_difference 33: note = May be 2'OH-thymidine
  misc_difference 34: note = May be 2'-O-Methyl-2'-deoxyadenosine
  misc_difference 35: note = May be deoxycytidine
  misc_difference 36: note = May be deoxycytidine
  misc_difference 37: note = May be 2'OH-thymidine
  misc_difference 38: note = May be 2'-O-Methyl-2'-deoxyguanosine
  misc_difference 39: note = May be deoxycytidine
  misc_difference 40: note = May be 2'-O-Methyl-2'-deoxyguanosine
  misc_difference 41: note = May be 2'-O-Methyl-2'-deoxycytidine
  misc_difference 42: note = May be 2'-O-Methyl-2'-deoxycytidine
  source 1..42: mol_type = other DNA

SEQ ID NO: 77 - RNA, 40 nt, synthetic construct; identical to SEQ ID NO: 28
  misc_feature 1..40: note = Synthetic C5 specific aptamer
  misc_difference 1: note = May have biotin conjugated to the 5' end
  misc_difference 1: note = May be 2'OH-adenosine
  misc_difference 2: note = May be 2'OH-guanosine
  misc_difference 3: note = May be 2'-fluoro-2'-deoxycytidine
  misc_difference 4: note = May be 2'-O-Methyl-2'-deoxyguanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'-fluoro-2'-deoxyuridine
  misc_difference 12: note = May be 2'-fluoro-2'-deoxycytidine
  misc_difference 13: note = May be 2'-fluoro-2'-deoxyuridine
  misc_difference 14: note = May be 2'-fluoro-2'-deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be 2'-fluoro-2'-deoxycytidine
  misc_difference 19: note = May be 2'OH-guanosine
  misc_difference 20: note = May be 2'-fluoro-2'-deoxycytidine
  misc_difference 21: note = May be 2'-fluoro-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'-fluoro-2'-deoxyuridine
  misc_difference 26: note = May be 2'-fluoro-2'-deoxycytidine
  misc_difference 27: note = May be 2'-fluoro-2'-deoxyuridine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxycytidine
  misc_difference 37: note = May be 2'-fluoro-2'-deoxyuridine
  misc_difference 38: note = May be 2'-O-Methyl-2'-deoxyguanosine
  misc_difference 39: note = May be 2'-fluoro-2'-deoxycytidine
  misc_difference 40: note = May be 2'-O-Methyl-2'-deoxyguanosine
  source 1..40: mol_type = other RNA

SEQ ID NO: 78 - DNA, 42 bp, synthetic construct; identical to SEQ ID NO: 73
  misc_feature 1..42: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-fluoro-2'-deoxycytidine
  misc_difference 4: note = May be 2'-O-Methyl-2'-deoxyguanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'-fluoro-2'-deoxyuridine
  misc_difference 12: note = May be deoxycytidine
  misc_difference 13: note = May be 2'OH-thymidine
  misc_difference 14: note = May be deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be deoxycytidine
  misc_difference 19: note = May be 2'OH-guanosine
  misc_difference 20: note = May be 2'-fluoro-2'-deoxycytidine
  misc_difference 21: note = May be 2'-fluoro-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'OH-thymidine
  misc_difference 26: note = May be deoxycytidine
  misc_difference 27: note = May be 2'OH-thymidine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxycytidine
  misc_difference 37: note = May be 2'-fluoro-2'-deoxyuridine
  misc_difference 38: note = May be 2'-O-Methyl-2'-deoxyguanosine
  misc_difference 39: note = May be 2'-fluoro-2'-deoxycytidine
  misc_difference 40: note = May be 2'-O-Methyl-2'-deoxyguanosine
  misc_difference 41: note = May be 2'-O-Methyl-2'-deoxycytidine
  misc_difference 42: note = May be 2'-O-Methyl-2'-deoxycytidine
  source 1..42: mol_type = other DNA

SEQ ID NO: 79 - RNA, 42 nt, synthetic construct
  misc_feature 1..42: note = Synthetic C5 specific aptamer
  misc_difference 1: note = May be 2'-O-Methyl-2'-deoxyguanosine
  misc_difference 2: note = May be 2'-O-Methyl-2'-deoxyguanosine
  misc_difference 3: note = May be 2'-fluoro-2'-deoxycytidine
  misc_difference 4: note = May be 2'-O-Methyl-2'-deoxyguanosine
  misc_difference 5: note = May be 2'-fluoro-2'-deoxycytidine
  misc_difference 6: note = May be 2'-fluoro-2'-deoxycytidine
  misc_difference 7: note = May be 2'OH-guanosine
  misc_difference 8: note = May be 2'-fluoro-2'-deoxycytidine
  misc_difference 9: note = May be 2'-O-Methyl-2'-deoxyguanosine
  misc_difference 10: note = May be 2'-O-Methyl-2'-deoxyguanosine
  misc_difference 11: note = May be 2'-fluoro-2'-deoxyuridine
  misc_difference 12: note = May be 2'-O-Methyl-2'-deoxycytidine
  misc_difference 13: note = May be 2'-O-Methyl-2'-deoxyuridine
  misc_difference 14: note = May be 2'-O-Methyl-2'-deoxycytidine
  misc_difference 15: note = May be 2'-O-Methyl-2'-deoxyadenosine
  misc_difference 16: note = May be 2'-O-Methyl-2'-deoxyguanosine
  misc_difference 17: note = May be 2'-O-Methyl-2'-deoxyguanosine
  misc_difference 18: note = May be 2'-O-Methyl-2'-deoxycytidine
  misc_difference 19: note = May be 2'OH-guanosine
  misc_difference 20: note = May be 2'-fluoro-2'-deoxycytidine
  misc_difference 21: note = May be 2'-fluoro-2'-deoxyuridine
  misc_difference 22: note = May be 2'-O-Methyl-2'-deoxyguanosine
  misc_difference 23: note = May be 2'-O-Methyl-2'-deoxyadenosine
  misc_difference 24: note = May be 2'-O-Methyl-2'-deoxyguanosine
  misc_difference 25: note = May be 2'-O-Methyl-2'-deoxyuridine
  misc_difference 26: note = May be 2'-O-Methyl-2'-deoxycytidine
  misc_difference 27: note = May be 2'-O-Methyl-2'-deoxyuridine
  misc_difference 28: note = May be 2'-O-Methyl-2'-deoxyguanosine
  misc_difference 29: note = May be 2'-O-Methyl-2'-deoxyadenosine
  misc_difference 30: note = May be 2'-O-Methyl-2'-deoxyguanosine
  misc_difference 31: note = May be 2'-fluoro-2'-deoxyuridine
  misc_difference 32: note = May be 2'-fluoro-2'-deoxyuridine
  misc_difference 33: note = May be 2'-fluoro-2'-deoxyuridine
  misc_difference 34: note = May be 2'OH-adenosine
  misc_difference 35: note = May be 2'-fluoro-2'-deoxycytidine
  misc_difference 36: note = May be 2'-fluoro-2'-deoxycytidine
  misc_difference 37: note = May be 2'-fluoro-2'-deoxyuridine
  misc_difference 38: note = May be 2'-O-Methyl-2'-deoxyguanosine
  misc_difference 39: note = May be 2'-fluoro-2'-deoxycytidine
  misc_difference 40: note = May be 2'-O-Methyl-2'-deoxyguanosine
  misc_difference 41: note = May be 2'-O-Methyl-2'-deoxycytidine
  misc_difference 42: note = May be 2'-O-Methyl-2'-deoxycytidine
  source 1..42: mol_type = other RNA
ggcgccgcggtctcaggcgctgagtctgagtttacctgcgcc

SEQ ID NO: 80 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'-O-Methyl-2'-deoxyadenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 81 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-O-Methyl-2'-deoxycytidine
  misc_difference 19: note = May be 2'-O-Methyl-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 82 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'OH-thymidine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 83 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-O-Methyl-2'-deoxycytidine
  misc_difference 19: note = May be 2'-O-Methyl-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'-O-Methyl-2'-deoxyadenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 84 - DNA, 39 bp, synthetic construct; identical to SEQ ID NO: 1
  misc_feature 1..39: note = Synthetic C5 specific aptamer
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-O-Methyl-2'-deoxycytidine
  misc_difference 19: note = May be 2'-O-Methyl-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'OH-thymidine
  misc_difference 30: note = May be 2'-fluoro-2'-deoxyuridine
  misc_difference 31: note = May be 2'-fluoro-2'-deoxyuridine
  misc_difference 32: note = May be 2'-O-Methyl-2'-deoxyadenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 85 - DNA, 39 bp, synthetic construct; identical to SEQ ID NO: 1
  misc_feature 1..39: note = Synthetic C5 specific aptamer
  misc_feature 1: note = May have a 20 kDa polyethylene glycol group attached via a hexylamine linker
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 86 - DNA, 39 bp, synthetic construct; identical to SEQ ID NO: 1
  misc_feature 1..39: note = Synthetic C5 specific aptamer
  misc_feature 1: note = May have a 30 kDa polyethylene glycol group attached via a hexylamine linker
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 87 - DNA, 39 bp, synthetic construct; identical to SEQ ID NO: 1
  misc_feature 1..39: note = Synthetic C5 specific aptamer
  misc_feature 1: note = May have a hexylamine terminal group
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 88 - DNA, 39 bp, synthetic construct; identical to SEQ ID NO: 1
  misc_feature 1..39: note = Synthetic C5 specific aptamer
  misc_feature 1: note = May have a 10 kDa polyethylene glycol group attached via a hexylamine linker
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 89 - RNA, 75 nt, synthetic construct
  misc_feature 1..75: note = Synthetic C5 specific aptamer
  source 1..75: mol_type = other RNA
gggagaggagagaacgttctaccttggtttggcacaggcatacatacgcaggggtcgatcgatcgatcatcgatg

SEQ ID NO: 90 - RNA, 32 nt, synthetic construct
  misc_feature 1..32: note = Synthetic C5 specific aptamer
  source 1..32: mol_type = other RNA
ccttggtttggcacaggcatacatacgcaggg

SEQ ID NO: 91 - RNA, 47 nt, synthetic construct
  misc_feature 1..47: note = Synthetic C5 specific aptamer
  source 1..47: mol_type = other RNA
cgttctaccttggtttggcacaggcatacatacgcaggggtcgatcg

SEQ ID NO: 92 - DNA, 39 bp, synthetic construct; identical to SEQ ID NO: 1
  misc_feature 1..39: note = Synthetic C5 specific aptamer
  misc_feature 1: note = May have a 40 kDa polyethylene glycol group attached via a hexylamine linker
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 39: note = May be an inverted orientation T (3'-3'-linked)
  source 1..39: mol_type = other DNA

SEQ ID NO: 93 - RNA, 38 nt, synthetic construct
  misc_feature 1..38: note = Synthetic C5 specific aptamer
  misc_feature 1: note = May have a 20 kDa polyethylene glycol group attached via a hexylamine linker
  misc_difference 1: note = May be 2'-fluoro-2'-deoxycytidine
  misc_difference 2: note = May be 2'-O-Methyl-2'-deoxyguanosine
  misc_difference 3: note = May be 2'-fluoro-2'-deoxycytidine
  misc_difference 4: note = May be 2'-fluoro-2'-deoxycytidine
  misc_difference 5: note = May be 2'OH-guanosine
  misc_difference 6: note = May be 2'-fluoro-2'-deoxycytidine
  misc_difference 7: note = May be 2'-O-Methyl-2'-deoxyguanosine
  misc_difference 8: note = May be 2'-O-Methyl-2'-deoxyguanosine
  misc_difference 9: note = May be 2'-fluoro-2'-deoxyuridine
  misc_difference 10: note = May be 2'-fluoro-2'-deoxycytidine
  misc_difference 11: note = May be 2'-fluoro-2'-deoxyuridine
  misc_difference 12: note = May be 2'-fluoro-2'-deoxycytidine
  misc_difference 13: note = May be 2'-O-Methyl-2'-deoxyadenosine
  misc_difference 14: note = May be 2'-O-Methyl-2'-deoxyguanosine
  misc_difference 15: note = May be 2'-O-Methyl-2'-deoxyguanosine
  misc_difference 16: note = May be 2'-fluoro-2'-deoxycytidine
  misc_difference 17: note = May be 2'OH-guanosine
  misc_difference 18: note = May be 2'-fluoro-2'-deoxycytidine
  misc_difference 19: note = May be 2'-fluoro-2'-deoxyuridine
  misc_difference 20: note = May be 2'-O-Methyl-2'-deoxyguanosine
  misc_difference 21: note = May be 2'-O-Methyl-2'-deoxyadenosine
  misc_difference 22: note = May be 2'-O-Methyl-2'-deoxyguanosine
  misc_difference 23: note = May be 2'-fluoro-2'-deoxyuridine
  misc_difference 24: note = May be 2'-fluoro-2'-deoxycytidine
  misc_difference 25: note = May be 2'-fluoro-2'-deoxyuridine
  misc_difference 26: note = May be 2'-O-Methyl-2'-deoxyguanosine
  misc_difference 27: note = May be 2'-O-Methyl-2'-deoxyadenosine
  misc_difference 28: note = May be 2'-O-Methyl-2'-deoxyguanosine
  misc_difference 29: note = May be 2'-fluoro-2'-deoxyuridine
  misc_difference 30: note = May be 2'-fluoro-2'-deoxyuridine
  misc_difference 31: note = May be 2'-fluoro-2'-deoxyuridine
  misc_difference 32: note = May be 2'OH-adenosine
  misc_difference 33: note = May be 2'-fluoro-2'-deoxycytidine
  misc_difference 34: note = May be 2'-fluoro-2'-deoxycytidine
  misc_difference 35: note = May be 2'-fluoro-2'-deoxyuridine
  misc_difference 36: note = May be 2'-O-Methyl-2'-deoxyguanosine
  misc_difference 37: note = May be 2'-fluoro-2'-deoxycytidine
  misc_difference 38: note = May be 2'-O-Methyl-2'-deoxyguanosine
  misc_feature 38: note = May have a 20 kDa polyethylene glycol group attached via a hexylamine linker
  source 1..38: mol_type = other RNA
cgccgcggtctcaggcgctgagtctgagtttacctgcg

SEQ ID NO: 94 - RNA, 80 nt, synthetic construct
  misc_feature 1..80: note = Synthetic C5 specific aptamer
  source 1..80: mol_type = other RNA
gggagaggagagaacgttctaccttggtttggcccaggcatatatacgcagggattgatccgttacgactagcatcgatg

SEQ ID NO: 95 - RNA, 79 nt, synthetic construct
  misc_feature 1..79: note = Synthetic C5 specific aptamer
  source 1..79: mol_type = other RNA
gggagaggagagaacgttctaccttaggttcgcactgtcatacatacacacgggcaatcggttacgactagcatcgatg

SEQ ID NO: 96 - RNA, 75 nt, synthetic construct
  misc_feature 1..75: note = Synthetic C5 specific aptamer
  misc_difference 34: note = n is a, c, g, or u
  misc_difference 43: note = n is a, c, g, or u
  source 1..75: mol_type = other RNA
gggagaggagagaacgttctaccttggtttggcncaggcatanatacgcacgggtcgatcggttacgactagcat

SEQ ID NO: 97 - protein, 126 aa, unidentified
  REGION 1..126: note = ankyrin binding domain
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA

SEQ ID NO: 98 - protein, 552 aa, synthetic construct
  REGION 1..552: note = recombinant human soluble VEGF receptor fusion protein
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

(19) United States
(12) Patent Application Publication
(10) Pub. No.: US 2025/0375467 A1
(43) Pub. Date: Dec. 11, 2025

(54) SUSTAINED RELEASE SILICA HYDROGEL COMPOSITES FOR TREATING OPHTHALMOLOGICAL CONDITIONS AND METHODS OF USING SAME

(71) Applicant: ASTELLAS US LLC, Northbrook, IL (US)

(72) Inventors: Gary COOK, Northbrook, IL (US); Pravin DUGEL, Northbrook, IL (US); Tatu ASSMUTH, Turku (FI)

(21) Appl. No.: 18/877,396

(22) PCT Filed: Jun. 29, 2023
(86) PCT No.: PCT/US2023/069364

§ 371 Date: Dec. 20, 2024

Related U.S. Application Data
(60) Provisional application No. 63/357,631, filed on Jun. 30, 2022.

(51) Int. Cl. A61K 31/7115; A61K 9/00; A61K 9/16

(52) U.S. Cl. CPC A61K 31/7115; A61K 9/0019; A61K 9/0051; A61K 9/1611

(57) ABSTRACT

Some embodiments relate to a sustained release silica hydrogel composite comprising an anti-complement agent and methods of using same to treat ophthalmological conditions. The anti-complement agent may be an anti-C5 agent comprising a C5-specific aptamer.

### CROSS-REFERENCE TO RELATED APPLICATIONS

This application is a national phase filing under 35 C.F.R. § 371 of and claims priority to PCT Patent Application No. PCT/US2023/069364, filed on Jun. 29, 2023, which claims the priority benefit under 35 U.S.C. § 119 of U.S. Provisional Patent Application No. 63/357,631, filed Jun. 30, 2022, which is incorporated by reference herein in its entirety for all purposes.

### REFERENCE TO AN ELECTRONIC SEQUENCE LISTING

The contents of the electronic sequence listing (OPHT_038_01WO_SeqList_ST26.xml; Size: 1,232,057 bytes; and Date of Creation: Jun. 27, 2023) are herein incorporated by reference in their entirety.

### TECHNICAL FIELD

The present disclosure relates to a sustained release silica hydrogel composite comprising an anti-complement agent and methods of using same to treat ophthalmological conditions.

### BACKGROUND

Age-related macular degeneration (“AMD”) is a disease characterized by progressive degenerative abnormalities in the macula, a region in the central portion of the retina. Age-related macular degeneration is a complex, gradually progressing disorder of the eye that leads to distortions and/or blind spots (scotoma), changes in dark adaptation (diagnostic of rod cell health), changes in color interpretation (diagnostic of cone cell health), a decrease in visual acuity, or irreversible blindness.

AMD is typically a disease of the elderly and is the leading cause of blindness in individuals >50 years of age in developed countries. In the United States, it is estimated that approximately 6% of individuals 65-74 years of age, and 20% of those older than 75 years of age, are affected with AMD. Because of increasing life expectancy in developed and developing [countries, the elderly portion of the general population is expected to increase at the greatest rate in coming decades. In the absence of adequate prevention or treatment measures, the number of cases of AMD with visual loss is expected to grow in parallel with the aging population.

Non-exudative AMD is the non-neovascular (“dry”) form of the disease (“dry AMD”). Dry AMD accounts for approximately 90% of all AMD cases. Dry AMD can be characterized by degeneration of the macula and, with continued progression over multiple years, may ultimately result in atrophy of the central retina associated with central vision loss. Dry AMD is a significant cause of moderate and severe loss of central vision and is bilateral in most patients. In dry AMD, thinning of the retinal pigment epithelial cells (RPE) in the macula develops, along with other age-related changes to the adjacent retinal tissue layers.

Once neovascularization arises in non-exudative AMD, the disease is referred to as exudative AMD, the neovascular (“wet”) form of the disease (“wet AMD”), with non-exudative AMD still present and potentially progressing in the patient. Wet AMD may cause sudden, often substantial, loss of central vision.

Recent advancements in imaging technology, specifically optical coherence tomography (“OCT”), and more specifically spectral domain optical coherence tomography (“SD-OCT”), have led to an ability to reproducibly and reliably measure morphological changes in the eyes of subjects exhibiting AMD disease states and to monitor the progression of disease over time. Such disease states include incomplete retinal pigment epithelium (“RPE”) and outer retinal atrophy (“iRORA”), risk factors for the progression to iRORA, complete RPE and outer retinal atrophy (“cRORA”), nascent geographic atrophy (“nGA”), and/or geographic atrophy (“GA”). See e.g., Guymer et al., “Incomplete Retinal Pigment Epithelial and Outer Retinal Atrophy in Age-Related Macular Degeneration: Classification of Atrophy Meeting Report 4”, Ophthalmology 2020; 127:394-409; see also Wu et al., “Optical Coherence Tomography-Defined Changes Preceding the Development of Drusen-Associated Atrophy in Age-Related Macular Degeneration”, Ophthalmology 2014; 121:2415-2422.

Administration of medicaments to treat ophthalmic conditions, such as the conditions described above, has been accomplished through intravitreal administration sometimes requiring monthly doses. There is a need for sustained release ophthalmic dosage forms comprising anti-complement agents that may be administered over longer intervals, which could result in greater patient comfort, satisfaction, and/or compliance. Such compositions must also meet and exhibit characteristics sufficient for administration through the ophthalmic route.

### SUMMARY

Provided herein is a sustained release silica hydrogel composite, the composite comprising: silica content in the range of 5-35% and anti-C5 agent in the range of 1-40%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

In some embodiments, the composite comprises silica content in the range of 5-35% and anti-C5 agent in the range of 5-40%. In some embodiments, the composite comprises silica content in the range of 5-30% and anti-C5 agent in the range of 1-5%, 5-10%, 10-15%, 15-20%, 20-25%, or 25-30%. In some embodiments, the composite comprises silica content in the range of 25-30% and anti-C5 agent in the range of 5-10%. In some embodiments, the composite comprises silica content of about 27.4% and anti-C5 agent of about 8%.

In some embodiments, the composite comprises silica microparticles dispersed in silica-sol hydrogel.

In some embodiments, the composite has a 2:1 ratio, 1:1 ratio, or 1:2 ratio of silica dissolution rate to anti-C5 agent dissolution rate.

In some embodiments of the composites provided herein, the anti-C5 agent is pegylated. In some embodiments of the composites provided herein, the anti-C5 agent is unpegylated.

Provided herein is a syringe comprising a sustained release silica hydrogel composite disclosed herein.

Provided herein is a method for ameliorating, treating or reducing the severity of a symptom of an ophthalmological condition in a subject in need thereof, the method comprising administering to the subject a sustained release silica hydrogel composite disclosed herein.

Provided herein is a method for preventing or delaying the progression of an ophthalmological condition in a subject in need thereof, the method comprising administering to the subject a sustained release silica hydrogel composite disclosed herein.

Provided herein is a method for treating or reducing the severity of an ophthalmological condition in a subject in need thereof, the method comprising administering to the subject a sustained release silica hydrogel composite disclosed herein.

In some embodiments of the methods provided herein, the ophthalmological condition is incomplete retinal pigment epithelial (RPE) and outer retinal atrophy, complete RPE and outer retinal atrophy, nascent geographic atrophy, geographic atrophy, or wet age-related macular degeneration.

In some embodiments of the methods provided herein, the sustained release silica hydrogel composite is administered to the subject by subconjunctival, retrobulbar, intracameral, sub-tenon, sub-retinal, suprachoroidal, or intravitreal injection. In some embodiments of the methods provided herein, the sustained release silica hydrogel composite is administered to the subject by intravitreal injection. In some embodiments of the methods provided herein, the sustained release silica hydrogel composite is administered to the subject by suprachoroidal injection.

In some embodiments of the methods provided herein, the sustained release silica hydrogel composite is administered to the subject at a dose of from about 0.3 mg/eye to about 5 mg/eye. In some embodiments of the methods provided herein, the sustained release silica hydrogel composite is administered to the subject at a dose of about 2 mg/eye.

In some embodiments of the methods provided herein, the sustained release silica hydrogel composite is administered to the subject at a frequency in which the duration between doses is at least about three months. In some embodiments of the methods provided herein, the sustained release silica hydrogel composite is administered to the subject at a frequency in which the duration between doses is about four months, about five months, or about six months.

Provided herein is a formulation comprising a population of microparticles, the microparticles comprising: silica content in the range of 10-70% and anti-C5 agent in the range of 5-50%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

In some embodiments, the microparticles comprise silica content in the range of 60-75% and anti-C5 agent in the range of 2.5-5.0%, 5-10%, 10-15%, 15-20%, 20-25%, or 25-30%. In some embodiments, the microparticles comprise silica content in the range of 60-72% and anti-C5 agent in the range of 2.5-25%. In some embodiments, the microparticles comprise silica content in the range of 64-68% and anti-C5 agent in the range of 15-19%.

In some embodiments of the formulations provided herein, the anti-C5 agent is pegylated. In some embodiments of the formulations provided herein, the anti-C5 agent is unpegylated.

### BRIEF DESCRIPTION OF THE DRAWINGS

FIG. 1A shows the particle size distributions for exemplary microparticle formulations.

FIG. 1B shows the D10, D50 and D90 values of formulations #01-#03.

FIG. 2A and FIG. 2B show in vitro dissolution data for formulations #04-#06.

FIG. 3A and FIG. 3B show in vitro dissolution data for formulations #07-#09. These experiments analyzed the effect of API (active pharmaceutical ingredient) load.

FIG. 4A and FIG. 4B show in vitro dissolution data for formulations #02-repeat, #10, and #11. These experiments analyzed the effect of batch size.

FIG. 5A shows the pH measurements of the microparticle and hydrogel formulations in Tables 1 and 2.

FIG. 5B shows the D10, D50 and D90 values of the microparticle formulation in Table 1.

FIG. 5C shows the particle size distribution of the microparticle formulation in Table 1.

FIG. 6A shows the SEM (scanning electron microscope) imaging of the microparticle formulations in Table 1 at a first image magnification.

FIG. 6B shows the SEM imaging of the microparticle formulations in Table 1 at a second image magnification.

FIG. 7 shows the in vitro release of API and silica matrix degradation in the microparticles #01-#03 in Table 3.

FIG. 8A shows the silica matrix degradation of the microparticle and hydrogel formulations in Tables 1 and 2.

FIG. 8B shows the API release of the microparticle and hydrogel formulations in Tables 1 and 2.

FIG. 8C shows a graph depicting the relationship between silica matrix degradation and API dissolution.

FIG. 9 shows a graph depicting Dutch belted rabbit ocular tissue exposure from the PK formulation. Solid lines from top to bottom are: Vitreous SR Depot (1 mg); Retina SR Depot (1 mg); RPE/Choroid SR Depot (1 mg). “IVT”=intravitreal. “SR”=sustained release.

FIG. 10A-FIG. 10D show data from an analysis of stability of the PK formulation at 2-8° C. after 8 weeks (8W). FIG. 10A shows silica degradation. FIG. 10B shows release of API. FIG. 10C shows Silica total content in hydrogel depots (wt.-%). FIG. 10D shows API total content in hydrogel depots (wt.-%).

FIG. 11A-FIG. 11D show data from an analysis of stability of the PK formulation at room temperature (RT) after 8 weeks (8W). FIG. 11A shows silica degradation. FIG. 11B shows release of API. FIG. 11C shows Silica total content in hydrogel depots (wt.-%). FIG. 11D shows API total content in hydrogel depots (wt.-%).

FIG. 12A-FIG. 12D show data from an analysis of aging of formulation #11. “1 W=1 week.

FIG. 13A and FIG. 13B show data from a rheology assessment of composite depots.

FIG. 14A-FIG. 14C show data from experiments analyzing the dissolution profile and microparticle size distribution of various formulations.

FIG. 15A-FIG. 15C show data from experiments analyzing the dissolution profile and microparticle size distribution of various formulations.

FIG. 16A-FIG. 16C show data from experiments analyzing the dissolution profile and microparticle size distribution of various formulations.

FIG. 17A-FIG. 17C show data from experiments analyzing the dissolution profile and microparticle size distribution of various formulations.

FIG. 18A-FIG. 18B show data from experiments analyzing the silica (FIG. 18A) and API (FIG. 18B) dissolution profiles of various formulations.

### DETAILED DESCRIPTION

One aspect of the present disclosure relates to a sustained release silica hydrogel composite comprising an anti-complement agent (such as an anti-C5 agent or an anti-C3 agent) and methods of using same to treat ophthalmological conditions. Sustained release silica hydrogel composites provided herein have favorable API delivery characteristics and stability properties and low build-up of residual matrix in the eye over time. These composites offer an unexpected advantage of a direct correlation between silica and API dissolution. Thus, the matrix that controls drug release does not remain in the eye long after the API is dissolved. Additionally, composites provided herein have a beneficial property of shear thinning of the composite depot formulation, which enables dosing using a narrow bore or gauge needle that is advantageous for intravitreal drug delivery.

### Anti-Complement Agents

Provided herein are sustained release silica hydrogel composites comprising an anti-complement agent. The term “anti-complement agent” refers to an agent that reduces, or inhibits, either partially or fully, the activity or production of a complement protein or a variant thereof.

In some embodiments, the anti-complement agent is an anti-C5 agent. The term “anti-C5 agent” refers to an agent that reduces, or inhibits, either partially or fully, the activity or production of a C5 complement protein or a variant thereof. An anti-C5 agent may reduce or inhibit the conversion of C5 complement protein into its component polypeptides C5a and C5b. Anti-C5 agents may also reduce or inhibit the activity or production of C5a and/or C5b.

In some embodiments, the anti-C5 agent is an anti-C5 aptamer. Aptamers are nucleic acid molecules having specific binding affinity to molecules through interactions other than classic Watson-Crick base pairing. Aptamers, like peptides generated by phage display or monoclonal antibodies (“mAbs”), are capable of specifically binding to selected targets and modulating the target's activity, e.g., through binding aptamers may block their target's ability to function. The aptamers may be unpegylated or pegylated. In some embodiments, the aptamers may contain one or more 2′ sugar modifications, such as 2′-O-alkyl (e.g., 2′-O-methyl or 2′-O-methoxyethyl) or 2′-fluoro modifications.

In some embodiments, the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

Further, illustrative C5 specific aptamers can also include the aptamers disclosed in PCT Publication No. WO 2007/103549, which is incorporated by reference in its entirety. For example, illustrative C5 specific aptamers can include the aptamers ARC185 (SEQ ID NO: 25), ARC186 (SEQ ID NO: 26), ARC188 (SEQ ID NO: 27), ARC189 (SEQ ID NO: 28), ARC243 (SEQ ID NO: 29), ARC244 (SEQ ID NO: 30), ARC250 (SEQ ID NO: 31), ARC296 (SEQ ID NO: 32), ARC297 (SEQ ID NO: 33), ARC330 (SEQ ID NO: 34), ARC331 (SEQ ID NO: 35), ARC332 (SEQ ID NO: 36), ARC333 (SEQ ID NO: 37), ARC334 (SEQ ID NO: 38), ARC411 (SEQ ID NO: 39), ARC412 (SEQ ID NO: 40), ARC413 (SEQ ID NO: 41), ARC414 (SEQ ID NO: 42), ARC415 (SEQ ID NO: 43), ARC416 (SEQ ID NO: 44), ARC417 (SEQ ID NO: 45), ARC418 (SEQ ID NO: 46), ARC419 (SEQ ID NO: 47), ARC420 (SEQ ID NO: 48), ARC421 (SEQ ID NO: 49), ARC422 (SEQ ID NO: 50), ARC423 (SEQ ID NO: 51), ARC424 (SEQ ID NO: 52), ARC425 (SEQ ID NO: 53), ARC426 (SEQ ID NO: 54), ARC427 (SEQ ID NO: 55), ARC428 (SEQ ID NO: 56), ARC429 (SEQ ID NO: 57), ARC430 (SEQ ID NO: 58), ARC431 (SEQ ID NO: 59), ARC432 (SEQ ID NO: 60), ARC433 (SEQ ID NO: 61), ARC434 (SEQ ID NO: 62), ARC435 (SEQ ID NO: 63), ARC436 (SEQ ID NO: 64), ARC437 (SEQ ID NO: 65), ARC438 (SEQ ID NO: 66), ARC439 (SEQ ID NO: 67), ARC440 (SEQ ID NO: 68), ARC457 (SEQ ID NO: 69), ARC458 (SEQ ID NO: 70), ARC459 (SEQ ID NO: 71), ARC473 (SEQ ID NO: 72), ARC522 (SEQ ID NO: 73), ARC523 (SEQ ID NO: 74), ARC524 (SEQ ID NO: 75), ARC525 (SEQ ID NO: 76), ARC532 (SEQ ID NO: 77), ARC543 (SEQ ID NO: 78), ARC544 (SEQ ID NO: 79), ARC550 (SEQ ID NO: 80), ARC551 (SEQ ID NO: 81), ARC552 (SEQ ID NO: 82), ARC553 (SEQ ID NO: 83), ARC554 (SEQ ID NO: 84), ARC657 (SEQ ID NO: 85), ARC658 (SEQ ID NO: 86), ARC672 (SEQ ID NO: 87), ARC706 (SEQ ID NO: 88), ARC913 (SEQ ID NO: 89), ARC874 (SEQ ID NO: 90), ARC954 (SEQ ID NO: 91), ARC1537 (SEQ ID NO: 92), ARC1730 (SEQ ID NO: 93), or a pharmaceutically acceptable salt thereof.

In some embodiments, the anti-C5 agent is an aptamer having the sequence of SEQ ID NO: 94, 95, or 96.

In some embodiments, ARC186 (SEQ ID NO: 26) can include 21 pyrimidine residues of ARC186 having 2′-fluoro modifications. The majority of purines (14 residues) have 2′-OMe modifications, except for three 2′-OH purine residues.

In some embodiments, an anti-C5 aptamer can also include different mixtures of 2′-fluoro and 2′-H modifications. In some embodiments, an anti-C5 aptamer anti-C5 aptamer is ARC330. ARC330 (SEQ ID NO: 34) contains seven 2′-H modifications, 14 pyrimidine residues with 2′-fluoro modifications, 14 purine residues with 2′-OMe modifications, and three 2′-OH purine residues.

In some embodiments, the aptamer may be pegylated, e.g., conjugated to a polyethylene glycol moiety (PEG) via a linker. The PEG moiety may have a molecular weight greater than about 10 kDa, such as a molecular weight of about 20 kDa, or about 30 kDa, or about 40 kDa, or about 50 kDa, or about 60 kDa. In some embodiments, the PEG moiety is conjugated via a linker to the 5′ end of the aptamer. In some embodiments, the PEG moiety conjugated to the 5′ end is a PEG moiety of about 40 kDa molecular weight. In some embodiments, the about 40 kDa PEG moiety is a branched PEG moiety. The branched about 40 kDa PEG moiety may be, for example, 1,3-bis(mPEG-[about 20 kDa])-propyl-2-(4′-butamide), or 2,3-bis(mPEG-[about 20 kDa])-propyl-1-carbamoyl. In some embodiments, the aptamer may be unpegylated.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

In some embodiments, the aptamer is a compound, ARC187, having the structure

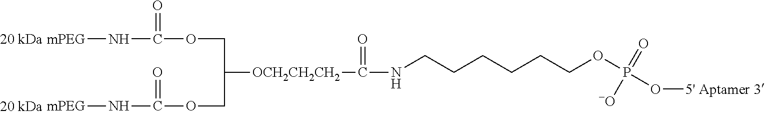

or a pharmaceutically acceptable salt thereof, where Aptamer=fCmGfCfCGfCmGmGfUfCfUfC mAmGmGfCGfCfUmGmAmGfUfCfUmGmAmGfUfUfUAfCfCfUmGfCmG-3T (SEQ ID NO: 1), in which fC and fU=2′-fluoro nucleotides, mG and mA=2′-OMe nucleotides, all other nucleotides are 2′-OH, and where 3T indicates an inverted deoxy thymidine. In some embodiments, each 20 kDa mPEG of the above structure has a molecular weight of about 20 kDa.

In some embodiments, the aptamer is a compound, ARC1905, having the structure set forth below:

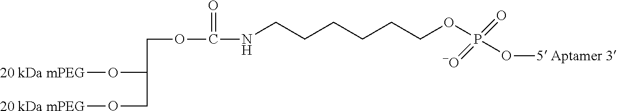

or a pharmaceutically acceptable salt thereof, where Aptamer=fCmGfCfCGfCmGmGfUfCfUfC mAmGmGfCGfCfUmGmAmGfUfCfUmGmAmGfUfUfUAfCfCfUmGfCmG-3T (SEQ ID NO: 1), in which fC and fU=2′-fluoro nucleotides, mG and mA=2′-OMe nucleotides, all other nucleotides are 2′-OH, and where 3T indicates an inverted deoxy thymidine. In some embodiments, each 20 kDa mPEG of the above structure has a molecular weight of about 20 kDa. As depicted in the figure, the above structure has a hexylamino linker.

In some embodiments, the anti-C5 agent comprises the active ingredient referred to as avacincaptad pegol (ACP). Avacincaptad pegol comprises the aptamer ARC1905.

In some embodiments, the anti-complement agent is an anti-C3 agent. The term “anti-C3 agent” refers to an agent that reduces, or inhibits, either partially or fully, the activity or production of a C3 complement protein or a variant thereof. An anti-C3 agent may reduce or inhibit the conversion of C3 complement protein into its component polypeptides C3a and C3b. Anti-C5 agents may also reduce or inhibit the activity or production of C3a and/or C3b.

In some embodiments, the anti-C3 agent is an anti-C3 aptamer. In some embodiments, the anti-C3 agent comprises the active ingredient referred to as pegcetacoplan.

Examples of a pharmaceutically acceptable salt include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts. The term “pharmaceutically acceptable salt” includes, but is not limited to, a hydrate of a compound provided herein and also may refer to a salt of an antagonist provided herein having an acidic functional group, such as, but not limited to, a carboxylic acid functional group or a hydrogen phosphate functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-, bis-, or tris-(2-hydroxyethyl)amine, 2-hydroxy-tert-butylamine, or tris-(hydroxymethyl)methylamine; N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

The anti-complement agent (e.g., anti-C5 agent or anti-C3 agent) can be administered as a component of a composition that further comprises a pharmaceutically acceptable carrier or vehicle, e.g., a pharmaceutical composition. The anti-C5 agent, for example, can be admixed with a suitable carrier substance, and is generally present in an amount of 1-95% by weight of the total weight of the composition. In some embodiments, the anti-complement agent (e.g., anti-C5 agent or anti-C3 agent) is present in an amount of 1-90%, 1-85%, 1-80%, 1-75%, 1-70%, 5-95%, 10-95%, 15-95%, 20-95%, 5-90%, 10-85%, 15-80%, or 20-75% by weight of the total weight of the composition. The composition may be provided in a dosage form that is suitable for injection, in particular suitable for injection directly in the eye (e.g., intravitreal injection). The composition may be in form of, for example, suspensions, emulsions, or solutions. The composition may include silica.

Formulations for injection include sterile aqueous or non-aqueous solutions, suspensions, emulsions, or gels. In some embodiments, a formulation for injection is a sol. In some embodiments, a formulation for injection is a hydrogel. A variety of aqueous carriers can be used, e.g., water, buffered water, saline, and the like. Such formulations may also contain excipients such as preserving agents, wetting agents, buffering agents, emulsifying agents, dispersing agents, and suspending agents.

In some embodiments, excipients for compositions that comprise the anti-complement agent (e.g., anti-C5 agent or anti-C3 agent) include, but are not limited to, buffering agents, nonionic surfactants, preservatives, tonicity agents, sugars, amino acids, and pH-adjusting agents. Suitable buffering agents include, but are not limited to, monobasic sodium phosphate, dibasic sodium phosphate, sodium acetate, sodium borate, and other buffers containing phosphate, acetate, borate, citrate, carbonate, and/or histidine. Suitable nonionic surfactants include, but are not limited to, polyoxyethylene sorbitan fatty acid esters such as polysorbate 20 and polysorbate 80. Suitable preservatives include, but are not limited to, benzyl alcohol, ascorbic acid/salts/esters, butylated hydroxytoluene, sulfites, and thiosulfate. Suitable tonicity agents include, but are not limited to sodium chloride, mannitol, and sorbitol. Suitable sugars include, but are not limited to, α,α-trehalose, glucose/dextrose, sucrose, mannitol, and sorbitol. Suitable amino acids include, but are not limited, to glycine and histidine. Suitable pH-adjusting agents include, but are not limited to, hydrochloric acid, acetic acid, and sodium hydroxide. In some embodiments, the pH-adjusting agent or agents are present in an amount effective to provide a pH of about 3 to about 8, about 6 to about 8, about 6.5 to about 8, about 4 to about 7, about 5 to about 6, about 6 to about 7, about 7 to about 8, or about 7 to about 7.5. In some embodiments, the pH-adjusting agent or agents are present in an amount effective to provide a pH of about 6.0 to about 6.5, about 6.5 to about 7.0, about 7.0 to about 7.5, or about 7.5 to about 8.0. In some embodiments, the pH-adjusting agent or agents are present in an amount effective to provide a pH of about 6.8 to about 7.8. In some embodiments, the compositions do not comprise a preservative. In some embodiments, the composition does not comprise an antimicrobial agent. In some embodiments, the composition does not comprise a bacteriostat.

### Silica

Silica (silicon dioxide, SiO₂) is a versatile material, which can be obtained naturally as well as prepared synthetically in many morphologies. Silica can be prepared/modified to many different structures by fuming or wet synthesis methods, which results in different properties both with respect to textural features and (surface) chemistry. For example, silica can be prepared via the sol-gel method. Sol-gel derived SiO₂ and other SiO₂-based materials can be commonly prepared from alkoxides, alkylalkoxides, aminoalkoxides or inorganic silicates that via hydrolysis form a sol that contains either partly hydrolyzed silica species and/or fully hydrolyzed silicic acid. Consequent condensation reactions of Si(OH)₄ containing species lead to formation of larger silica species with increasing amount of siloxane bonds. These silica species oligomerize/polymerize, and small particles are formed, turning the reaction solution to a sol.

Silica prepared by sol-gel method can be processed to three-dimensional structures by casting (e.g., monolithic rods), spinning (fibers), by dipping/draining/spinning (coatings) or by preparing particles of different size. In some embodiments, particles are prepared either by spray-drying that result in particles or spheres mostly on micrometer scale or by letting the particles grow in size and number in the sol in alkaline conditions, which results in colloidal silica dispersion, i.e., submicron, nanoscale particles in a solution. The liquids in the colloidal dispersion can be evaporated and the formed powder of colloidal particles is typically washed and dried several times. Particles are sometimes prepared also by grinding, e.g., monoliths to desired size. All the conventional sol-gel processing methods involve a step, where the structure is dried and/or heat-treated to some extent and the amount of solutions/solvents such as water and alcohols are more or less diminished. In some embodiments, silica particles are prepared by spray drying or liquid phase synthesis, by chopping spun or drawn silica fibers, by molding or casting silica monoliths and, when necessary for obtaining defined particle size, by crushing molded or cast silica monoliths.

In some embodiments, the gel can be a homogeneous mixture of at least one solid phase and one liquid phase, i.e., a colloidal dispersion, where solid phase(s), e.g., silica as such and/or as partly or fully hydrolyzed, is the continuous phase and the liquid(s), e.g., water, ethanol and residuals of silica precursors, is homogeneously dispersed in the structure. The gel is viscoelastic and the elastic properties dominate, which is indicated by rheological measurements under small angle oscillatory shear that the storage modulus (elastic component), G′ is greater than the loss modulus (viscous component), G″ (G′>×G″). The gel is non-flowing at rest but flowing under shear. In some embodiments, G′>2×G″, G′>3×G″, G′>4×G″, G′>5×G″, G′>6×G″, G′>7×G″, G′>8×G″, G′>9×G″, G′>10×G″, G′>25×G″, G′>50×G″, G′>75×G″, G′>100×G″, G′>250×G″, G′>500×G″, G′>750×G″, or G′>1000×G″.

In some embodiments, the sol can be a homogeneous mixture of at least one liquid phase and one solid phase, i.e., a colloidal dispersion, where the liquid phase(s), e.g., water, ethanol and residuals of silica precursors, is the continuous phase and the solid phase(s), e.g., colloidal particles of silica and/or as partly or fully hydrolyzed silica and/or aggregates of said particles are homogeneously dispersed in the said liquid phase characterized in that the sol has clear flow properties and the liquid phase is dominating.

### Composition Comprising an Anti-Complement Agent and Silica

In some embodiments, an exemplary composition for treating ophthalmological conditions can be a microparticle composition comprising silica content in the range of 30-90%, with preferred embodiments of 30-40%, 40-50%, 30-65%, 50-60%, 60-70%, 70-80%, or 80-90%, anti-C5 agent content in the range of 1-60%, with preferred embodiments of 1-10%, 10-20%, 20-30%, 20-55%, 30-40%, 40-50%, or 50-60%, and residuals of the silica content precursors in the range of 1-40%, preferred embodiments of 1-5%, 5-10%, 10-15%, 15-20%, 20-25%, 25-30%, 30-35%, 35-40%, 40-45%, or 45-50%. In some embodiments, the microparticle composition comprises silica content in the range of 64-68%, anti-C5 agent content in the range of 15-19%, and residuals of the silica content precursors in the range of 14-18%. In some embodiments, the microparticle composition comprises silica content in the range of 30-65%, and anti-C5 agent content in the range of 20-55%. In some embodiments, the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine. In some embodiments, the residuals comprise one or more products from the silica precursor. In certain embodiments, the silica precursor is tetraethyl orthosilicate ethyl silicate (TEOS), and the hydrolysis product is ethanol.

In some embodiments, a sustained release silica hydrogel composite can comprise silica content in the range of 10-50%, with preferred embodiments of 10-30%, 20-50%, 20-25%, 25-30%, 30-35%, 35-40%, 40-45%, or 45-50%, anti-C5 agent content in the range of 1-30% or 1-50%, with preferred embodiments 1-5%, 5-10%, 5-30%, 10-15%, 15-20%, 20-25%, or 25-30%, and water and residuals of the silica content precursors are in the range of 40-80%, with preferred embodiments of 40-50%, 50-60%, 55-70%, 60-70%, or 70-80%. In some embodiments, the silica composite comprises silica content in the range of 24-34%, anti-C5 agent content in the range of 6-9%, and water and residuals of the silica content precursors are in the range of 61-69%. In some embodiments, the silica composite comprises silica content in the range of 10-30%, anti-C5 agent content in the range of 5-30%, and water and residuals of the silica content precursors are in the range of 55-70%. In some embodiments, the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine. In some embodiments, the residuals comprise water and one or more products from the silica precursor. In some embodiments, the silica precursor is TEOS and the hydrolysis product is ethanol. In some embodiments, the silica hydrogel composite is a silica-based microparticle containing an anti-C5 agent, which is one of dispersed, suspended, or contained within a silica-based hydrogel.

Provided herein is a sustained release silica hydrogel composite, the composite comprising: silica content in the range of 5-35% and anti-C5 agent in the range of 1-40%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

In some embodiments, the composite comprises silica content in the range of 5-35% and anti-C5 agent in the range of 5-40%. In some embodiments, the composite comprises silica content in the range of 5-30% and anti-C5 agent in the range of 1-5%, 5-10%, 10-15%, 15-20%, 20-25%, or 25-30%. In some embodiments, the composite comprises silica content in the range of 25-30% and anti-C5 agent in the range of 5-10%. In some embodiments, the composite comprises silica content of about 27.4% and anti-C5 agent of about 8%.

In some embodiments, a sustained release silica hydrogel composite has a 2:1 ratio, 1:1 ratio, or 1:2 ratio of silica dissolution rate to anti-C5 agent dissolution rate. In some embodiments, a sustained release silica hydrogel composite has a 1:1 ratio of silica dissolution rate to anti-C5 agent dissolution rate.

In some embodiments, an exemplary composition can be a microparticle composition comprising silica content in the range of 5-70%, or 40-90%, with preferred embodiments of 40-50%, 50-60%, 60-70%, 70-80%, or 80-90%, anti-C3 agent content in the range of 1-60%, with preferred embodiments of 1-10%, 1-40%, 10-20%, 20-30%, 30-40%, 40-50%, or 50-60%, and residuals of the silica content precursors in the range of 1-40%, preferred embodiments of 1-5%, 5-10%, 10-15%, 15-20%, 20-25%, 25-30%, 30-35%, 35-40%, 40-45%, or 45-50%.

Provided herein is a formulation comprising a population of microparticles, the microparticles comprising: silica content in the range of 10-70% and anti-C5 agent in the range of 5-50%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

In some embodiments, the microparticles comprise silica content in the range of 60-75% and anti-C5 agent in the range of 2.5-5.0%, 5-10%, 10-15%, 15-20%, 20-25%, or 25-30%. In some embodiments, the microparticles comprise silica content in the range of 60-72% and anti-C5 agent in the range of 2.5-25%. In some embodiments, the microparticles comprise silica content in the range of 64-68% and anti-C5 agent in the range of 15-19%.

In some embodiments, a sustained release silica hydrogel composite comprises silica microparticles dispersed in silica-sol hydrogel. In some embodiments, a sustained release silica hydrogel composite is a depot formulation, which is microparticles suspended in silica-sol hydrogel. In some embodiments, a depot formulation provided herein exhibits shear thinning. The depot formulation can be filled into a container closure system, for example, a dosing syringe. Provided herein is a syringe comprising a sustained release silica hydrogel composite disclosed herein.

### Early-Stage AMD

Provided herein is a method for ameliorating, treating or reducing the severity of a symptom of an ophthalmological condition in a subject in need thereof, the method comprising administering to the subject a sustained release silica hydrogel composite provided herein. Also provided herein is a method for preventing or delaying the progression of an ophthalmological condition in a subject in need thereof, the method comprising administering to the subject a sustained release silica hydrogel composite provided herein. Also provided herein is a method for treating or reducing the severity of an ophthalmological condition in a subject in need thereof, the method comprising administering to the subject a sustained release silica hydrogel composite provided herein. In some embodiments, an ophthalmological condition is iRORA, cRORA, nGA, GA, and/or wet AMD.

In some embodiments, the present disclosure relates to methods and compositions useful for subjects with incomplete retinal pigment epithelium (RPE) and outer retinal atrophy (“iRORA”). iRORA is an ophthalmological disease, disorder, and/or condition characterized by the following three features, which are vertically aligned and determined by OCT: (1) a region of signal hypertransmission into the choroid, (2) a corresponding zone of attenuation or disruption of the RPE, and (3) evidence or signs of overlying photoreceptor degeneration. Evidence or signs of overlying photoreceptor degeneration include subsidence of the inner nuclear layer (“INL”) and outer plexiform layer (“OPL”), presence of a hyporeflective wedge in the Henle fiber layer (“HFL”), thinning of the outer nuclear layer (“ONL”), disruption of the external limiting membrane (“ELM”), and disintegrity of the ellipsoid zone (“EZ”). iRORA should not be used to refer to an RPE tear.

In some embodiments, the present disclosure relates to methods and compositions useful for subjects with risk factors for the progression to iRORA. A subject who exhibits risk factors for the progression to iRORA exhibits some, but not all, of the signs of iRORA, as described above. In addition, a subject exhibiting high-risk drusen and risk factors for the progression to iRORA may also exhibit hyperreflective foci, heterogeneous internal reflectivity of drusen, and/or subretinal drusenoid deposits. Determination of whether a subject has risk factors for the progression to iRORA is also accomplished with multimodal imaging, which includes but is not limited to OCT. A subject with risk factors for the progression to iRORA may progress to iRORA, cRORA, nGA, GA and/or wet AMD.

Risk factors are known in the art and include, e.g., hypertension, obesity, atherosclerosis, focal deposition of acellular detritus between the retinal pigment epithelium (RPE), family history of AMD, including a genetic risk, smoking, high body mass index, high-fat diet, low intake of antioxidants and zinc, previous cataract surgery, history of cardiovascular disease, higher plasma fibrinogen, and/or diabetes (see, e.g., Garcia-Layana et al., Clinical Interventions in Aging 2017:12 1579-1587, the contents of which are incorporated herein by reference in their entirety).

In some embodiments, the present disclosure relates to methods and compositions useful for subjects with cRORA. cRORA is an ophthalmological disease, disorder, and/or condition meeting the requirements of iRORA and further requiring a change in the areas of RPE, hypertransmission having a diameter of at least 250 μm on the OCT B-scan, and evidence of photoreceptor loss. iRORA may progress to cRORA, nGA, GA and/or wet AMD.

In some embodiments, the present disclosure relates to methods and compositions useful for subjects with nGA. nGA is an ophthalmological disease, disorder, and/or condition characterized by: (i) the subsidence of the inner nuclear layer (INL) and outer plexiform later (OPL), and (ii) a hyporeflective wedge-shaped band within the OPL, including within the Henle fiber layer. nGA may also be accompanied by RPE disturbance and increased signal hypertransmission into the choroid. In addition, features frequently present in subsidence of the OPL and INL may include disruption of the inner segment ellipsoid (“ISe”), a break in the ELM, and traces of increased signal transmission below Bruch's membrane. Furthermore, features frequently present with a hyporeflective wedge-shaped band include a vortex-like subsidence of OPL and INL, drusen regression, and traces of increased signal transmission below RPE. The onset of nGA may also be accompanied by, or preceded by, the regression of some or all drusen, resulting in the overlying retinal layers undergoing characteristic changes in progressive atrophy. nGA may also be associated with, and/or occur simultaneously with, an iRORA disease state. A subject exhibiting nGA may have previously exhibited risk factors for the progression to iRORA and may subsequently exhibit cRORA and/or GA.

In some embodiments, the present disclosure includes methods of administration to subjects with high-risk drusen. High-risk drusen refers to drusen associated with a high risk of AMD and/or a high risk of disease progression from an earlier-stage AMD to a later-stage AMD. High-risk drusen may have any of the following characteristics. For example, high-risk drusen may be characterized by the presence of at least one druse with a diameter of at least 250 μm observed on fundus biomicroscopy or color fundus photography and/or a total volume of drusen of at least 0.03 mm³ as measured by SD-OCT within a 3 mm diameter circle centered on the fovea. In some embodiments, high-risk drusen may have a diameter of at least 300 μm and exist within a 500 μm diameter circle centered on the fovea.

In addition, high-risk drusen may be characterized by other morphological features. Furthermore, high-risk drusen may be characterized in terms of maximum lesion height and diameter, lesion internal reflectivity, presence and extent of overlying intraretinal hyperreflective foci, and choroidal thickness both subfoveally and below drusen. In addition, high-risk drusen may exhibit hyperreflective foci overlying the drusen, heterogeneous internal reflectivity of drusen, or choroidal thickness less than 135 μm below the drusen baseline. In addition, high-risk drusen may be soft, large, indistinct, and/or confluent.

In some embodiments, the subject exhibits hyperpigmentation or hypopigmentation. In some embodiments, increasing hyperpigmentation is another way to signify disease progression. In some embodiments, hypopigmentation is associated with specific disease states.

SD-OCT specifically provides a reliable and reproducible method for measuring drusen morphology over time as well as other characteristic features of AMD. Additionally, SD-OCT algorithms are available in order to quantify drusen characteristics. Such algorithms can be fully-automated and reliably report drusen load, drusen volume and area and morphological changes over time using cube root and square root transformations, respectively. Advancements of imaging with the use of SD-OCT and color fundus imaging has made it possible to study and measure the morphology of drusen by providing three-dimensional, geometric assessment. SD-OCT imaging has also allowed for multimodal imaging and has identified other macular features that increase the risk of vision loss, including decreased internal reflectivity of drusen (identified as calcified drusen), intraretinal hyperreflective foci, and subretinal drusenoid deposits. Instruments used for SD-OCT are known in the art, such as the Cirrus HD-OCT.

### Administration and Dosage

The dosage of the anti-complement agent (e.g., anti-C5 agent) for administration to the eye may be about 0.1 mg/eye to about 5 mg/eye, about 0.3 mg/eye to about 5 mg/eye, about 0.5 mg/eye to about 3 mg/eye, about 1 mg/eye to about 3 mg/eye, about 1 mg to about 4 mg/eye, or about 2 mg/eye to about 4 mg/eye. In some embodiments, the dosage of the anti-C5 agent for administration to the eye may be about 0.3 mg/eye, or about 0.5 mg/eye, or about 0.75 mg/eye, or about 1 mg/eye, or about 1.25 mg/eye, or about 1.50 mg/eye, or about 1.75 mg/eye, or about 2 mg/eye, or about 2.25 mg/eye, or about 2.50 mg/eye, or about 2.75 mg/eye, or about 3 mg/eye, or about 3.25 mg/eye, or about 3.50 mg/eye, or about 3.75 mg/eye, or about 4 mg/eye.

In some embodiments, the dosage of the anti-complement agent (e.g., anti-C5 agent) for administration to the eye may be from about 0.3 mg/eye to about 5 mg/eye. In some embodiments, the dosage of the anti-complement agent (e.g., anti-C5 agent) for administration to the eye may be about 2 mg/eye.

The dosage of the anti-complement agent (e.g., anti-C5 agent) for administration to the eye may be an oligonucleotide equivalent dose of about 100-200, about 200-400, about 400-600, about 600-800, or about 800-1000 μg.

The daily drug dose provided by the silica composite (e.g., microparticles and silica hydrogel) may be about 0.1-100 μg, or about 0.1-0.5 μg, or about 0.5-1.0 μg, or about 1-5 μg, or about 5-10 μg, or about 10-20 μg, or about 20-30 μg, or about 30-40 μg, or about 40-50 μg, or about 50-60 μg, or about 60-70 μg, or about 70-80 μg, or about 80-90 μg, or about 90-100 μg.

The dosage of the anti-complement agent (e.g., anti-C5 agent) for administration to the eye may be 10-100 ng/day, 100-1000 ng/day, 1-10 μg/day, and 10-100 μg/day. In some embodiments, the dosage of the anti-complement agent (e.g., anti-C5 agent) for administration to the eye may range from 0.5 μg/day to 15 μg/day.

The silica composite (e.g., microparticles and silica hydrogel) may be administered once every 8 weeks, once every 9 weeks, once every 10 weeks, once every 11 weeks, once every 12 weeks, once every 13 weeks, once every 14 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 6 months, once every 7 months, once every 8 months, once every 9 months, once every 10 months, once every 11 months, or once every 12 months. The dosages may be administered once every two months, once every three months, once every four months, once every five months, or once every six months.

In some embodiments, the silica composite (e.g., microparticles and silica hydrogel) may be administered intra- or peri-ocularly, for example by subconjunctival, retrobulbar, intracameral, sub-tenon, sub-retinal, suprachoroidal, or intravitreal injection. In some embodiments, the silica composite is administered by intravitreal injection. In some embodiments, the silica composite is administered by suprachoroidal injection. The silica composite may be in the form of a depot.

In some embodiments, a dosing regimen comprising a loading phase and maintenance phase may be administered. In some embodiments, the silica composite (e.g., microparticles and silica hydrogel) may be administered in a dosing regimen comprising a loading phase and maintenance phase. In some embodiments, the loading phase may comprise administering the anti-C5 agent, while the maintenance phase may comprise administering the silica composite (e.g., microparticles and silica hydrogel). In some embodiments, the anti-C5 agent is avacincaptad pegol. In some embodiments, the loading phase may comprise administering avacincaptad pegol at a different dosage, at a different frequency, or a combination thereof, as compared to the avacincaptad pegol in the silica composite during maintenance phase. For instance, the loading phase may comprise administering the avacincaptad pegol at a dose of about 0.3 mg/eye, 0.5 mg/eye, or about 1 mg/eye, or about 2 mg/eye, or about 3 mg/eye, or about 4 mg/eye; and the maintenance phase may comprise administering the avacincaptad pegol in the silica composite at a dose that is a percentage of, or greater than, the dose of the avacincaptad pegol of the loading phase, such as about 10%, or about 20%, or about 25%, or about 30%, or about 33%, or about 40%, or about 50%, or about 60%, or about 67%, or about 70%, or about 75%, or about 80%, or about 90%, or about 100%, or about 125%, or about 150%, or about 175%, or about 200%, or about 225%, or about 250%, or about 275%, or about 300%, or about 325%, or about 350%, or about 375%, or about 400%, of the dose of the avacincaptad pegol of the loading phase. Alternatively, or in addition, the loading phase may comprise administering avacincaptad pegol at a frequency in which the duration between doses is one week, two weeks, three weeks, four weeks, one month, five weeks, six weeks, seven weeks, eight weeks, two months, nine weeks, 10 weeks, 11 weeks, 12 weeks, three months, four months, five months, or six months; and the maintenance phase may comprise administering the avacincaptad pegol in the silica composite at a frequency in which the duration between doses is a percentage of, or greater than, the duration between doses of the loading phase, such as about 10%, or about 20%, or about 25%, or about 30%, or about 33%, or about 40%, or about 50%, or about 60%, or about 67%, or about 70%, or about 75%, or about 80%, or about 90%, or about 100%, or about 125%, or about 150%, or about 175%, or about 200%, or about 225%, or about 250%, or about 275%, or about 300%, or about 325%, or about 350%, or about 375%, or about 400%, of the duration between doses of the loading phase. In some embodiments, the loading phase may last a duration of about 1 week, about 2 weeks, about 3 weeks, about 4 weeks, about 1 month, about five weeks, about six weeks, about seven weeks, about eight weeks, about 2 months, about nine weeks, about 10 weeks, about 11 weeks, about 12 weeks, about 3 months, about 4 months, about 5 months, about 6 months, about 7 months, about 8 months, about 9 months, about 12 months, about 15 months, about 18 months, about 21 months, or about 24 months; and the maintenance phase may begin concurrently with, any time within the, or at the conclusion of the loading phase.

In certain embodiments, avacincaptad pegol may be administered in a dosing regimen comprising a loading phase that comprises a dose of about 2 mg/eye administered once a month for a duration of up to one year, followed by a maintenance phase that comprises a dose of the avacincaptad pegol in the silica composite of about 0.1 mg/eye, or about 0.3 mg/eye, or about 0.5 mg/eye, or about 0.75 mg/eye, or about 1 mg/eye, or about 1.25 mg/eye, or about 1.50 mg/eye, or about 1.75 mg/eye, or about 2 mg/eye, or about 2.25 mg/eye, or about 2.50 mg/eye, or about 2.75 mg/eye, or about 3 mg/eye, or about 3.25 mg/eye, or about 3.50 mg/eye, or about 3.75 mg/eye, or about 4 mg/eye, administered once every 8 weeks, once every 9 weeks, once every 10 weeks, once every 11 weeks, once every 12 weeks, once every 13 weeks, once every 14 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, or once every 26 weeks. In certain embodiments, avacincaptad pegol may be administered in a dosing regimen comprising a loading phase that comprises a dose of about 4 mg/eye administered once a month for a duration of up to one year, followed by a maintenance phase that comprises a dose of the avacincaptad pegol in the silica composite of about 0.3 mg/eye, or about 0.5 mg/eye, or about 0.75 mg/eye, or about 1 mg/eye, or about 1.25 mg/eye, or about 1.50 mg/eye, or about 1.75 mg/eye, or about 2 mg/eye, or about 2.25 mg/eye, or about 2.50 mg/eye, or about 2.75 mg/eye, or about 3 mg/eye, or about 3.25 mg/eye, or about 3.50 mg/eye, or about 3.75 mg/eye, or about 4 mg/eye, administered once every 8 weeks, once every 9 weeks, once every 10 weeks, once every 11 weeks, once every 12 weeks, once every 13 weeks, once every 14 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, or once every 26 weeks. In certain embodiments, the avacincaptad pegol may be administered in a dosing regimen comprising a loading phase that comprises a dose of about 2 mg/eye administered once a month for a duration of about 6 months, followed by a maintenance phase that comprises a dose of the avacincaptad pegol in the silica composite of about 0.3 mg/eye, or about 0.5 mg/eye, or about 0.75 mg/eye, or about 1 mg/eye, or about 1.25 mg/eye, or about 1.50 mg/eye, or about 1.75 mg/eye, or about 2 mg/eye, or about 2.25 mg/eye, or about 2.50 mg/eye, or about 2.75 mg/eye, or about 3 mg/eye, or about 3.25 mg/eye, or about 3.50 mg/eye, or about 3.75 mg/eye, or about 4 mg/eye, administered once every 8 weeks, once every 9 weeks, once every 10 weeks, once every 11 weeks, once every 12 weeks, once every 13 weeks, once every 14 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, or once every 26 weeks.

The amount of the silica composite (e.g., microparticles and silica hydrogel) administered to the subject can range from about 10 to about 2000 μL, or from about 25 to about 2000 μL. In some embodiments, the silica composite may be administered intra- or peri-ocularly, for example by subconjunctival, retrobulbar, intracameral, sub-tenon, sub-retinal, suprachoroidal, or intravitreal injection. In some embodiments, the silica composite may be administered by intravitreal injection. In some embodiments, the silica composite may be administered by suprachoroidal injection.

In the present description, any concentration range, percentage range, ratio range, or integer range is to be understood to include the value of any integer within the recited range and, when appropriate, fractions thereof (such as one tenth and one hundredth of an integer), unless otherwise indicated.

### Numbered Embodiments

Notwithstanding the appended claims, the disclosure sets forth the following numbered embodiments:

Embodiment 1. A sustained release silica hydrogel composite, the composite comprising: silica content in the range of 5-35% and anti-C5 agent in the range of 1-40%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

Embodiment 2. The sustained release silica hydrogel composite of embodiment 1, wherein the composite comprises silica content in the range of 5-35% and anti-C5 agent in the range of 5-40%.

Embodiment 3. The sustained release silica hydrogel composite of embodiment 1, wherein the composite comprises silica content in the range of 5-30% and anti-C5 agent in the range of 1-5%, 5-10%, 10-15%, 15-20%, 20-25%, or 25-30%.

Embodiment 4. The sustained release silica hydrogel composite of embodiment 1, wherein the composite comprises silica content in the range of 25-30% and anti-C5 agent in the range of 5-10%.

Embodiment 5. The sustained release silica hydrogel composite of embodiment 1, wherein the composite comprises silica content of about 27.4% and anti-C5 agent of about 8%.

Embodiment 6. The sustained release silica hydrogel composite of any one of embodiments 1-5, wherein the composite comprises silica microparticles dispersed in silica-sol hydrogel.

Embodiment 7. The sustained release silica hydrogel composite of any one of embodiments 1-6, wherein the composite has a 2:1 ratio, 1:1 ratio, or 1:2 ratio of silica dissolution rate to anti-C5 agent dissolution rate.

Embodiment 8. The sustained release silica hydrogel composite of any one of embodiments 1-7, wherein the anti-C5 agent is pegylated.

Embodiment 9. The sustained release silica hydrogel composite of any one of embodiments 1-7, wherein the anti-C5 agent is unpegylated.

Embodiment 10. A syringe comprising the sustained release silica hydrogel composite of any one of embodiments 1-9.

Embodiment 11. A method for ameliorating, treating or reducing the severity of a symptom of an ophthalmological condition in a subject in need thereof, the method comprising administering to the subject the sustained release silica hydrogel composite of any one of embodiments 1-9.

Embodiment 12. A method for preventing or delaying the progression of an ophthalmological condition in a subject in need thereof, the method comprising administering to the subject the sustained release silica hydrogel composite of any one of embodiments 1-9.

Embodiment 13. A method for treating or reducing the severity of an ophthalmological condition in a subject in need thereof, the method comprising administering to the subject the sustained release silica hydrogel composite of any one of embodiments 1-9.

14. The method of any one of embodiments 11-13, wherein the ophthalmological condition is incomplete retinal pigment epithelial (RPE) and outer retinal atrophy, complete RPE and outer retinal atrophy, nascent geographic atrophy, geographic atrophy, or wet age-related macular degeneration.

Embodiment 15. The method of any one of embodiments 11-14, wherein the sustained release silica hydrogel composite is administered to the subject by subconjunctival, retrobulbar, intracameral, sub-tenon, sub-retinal, suprachoroidal, or intravitreal injection.

Embodiment 16. The method of any one of embodiments 11-14, wherein the sustained release silica hydrogel composite is administered to the subject by intravitreal injection.

Embodiment 17. The method of any one of embodiments 11-14, wherein the sustained release silica hydrogel composite is administered to the subject by suprachoroidal injection.

Embodiment 18. The method of any one of embodiments 11-17, wherein the sustained release silica hydrogel composite is administered to the subject at a dose of from about 0.3 mg/eye to about 5 mg/eye.

Embodiment 19. The method of any one of embodiments 11-17, wherein the sustained release silica hydrogel composite is administered to the subject at a dose of about 2 mg/eye.

Embodiment 20. The method of any one of embodiments 11-19, wherein the sustained release silica hydrogel composite is administered to the subject at a frequency in which the duration between doses is at least about three months.

Embodiment 21. The method of any one of embodiments 11-19, wherein the sustained release silica hydrogel composite is administered to the subject at a frequency in which the duration between doses is about four months, about five months, or about six months.

Embodiment 22. A formulation comprising a population of microparticles, the microparticles comprising: silica content in the range of 10-70% and anti-C5 agent in the range of 5-50%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUA fCfCfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

Embodiment 23. The formulation of embodiment 22, wherein the microparticles comprise silica content in the range of 60-75% and anti-C5 agent in the range of 2.5-5.0%, 5-10%, 10-15%, 15-20%, 20-25%, or 25-30%.

Embodiment 24. The formulation of embodiment 22, wherein the microparticles comprise silica content in the range of 60-72% and anti-C5 agent in the range of 2.5-25%.

Embodiment 25. The formulation of embodiment 22, wherein the microparticles comprise silica content in the range of 64-68% and anti-C5 agent in the range of 15-19%.

Embodiment 26. The formulation of any one of embodiments 22-25, wherein the anti-C5 agent is pegylated.

Embodiment 27. The formulation of any one of embodiments 22-25, wherein the anti-C5 agent is unpegylated.

The disclosure will be further clarified by the following examples, which are intended to be purely exemplary of the disclosure and in no way limiting.

### Example 1A: Preparation of Silica Microparticles Comprising an Anti-C5 Agent Employing a Semi-Batch Reactor Process

The manufacture of silica microparticles was prepared using the following general procedure: preparation of anti-C5 agent (avacincaptad pegol) and NaOH solutions, preparation of silica sol by TEOS hydrolysis in a batch reactor, mixing of the component solutions (avacincaptad pegol solution, NaOH solution and silica sol) in a semi-batch reactor, and spray drying.

To prepare an avacincaptad pegol-water solution, 538.2 mg of avacincaptad pegol was weighed and dissolved in 35.9 ml of milli-Q water to obtain a 15 mg/ml solution.

TEOS was hydrolyzed in a batch reactor. The preparation of silica microparticles with 15% (w/w) encapsulated avacincaptad pegol was begun with the manufacture of silica sol. The sol is prepared by mixing the silica precursor, tetraethyl orthosilicate (TEOS, Sigma Aldrich), with milli-Q water (Merck Millipore) and 0.1 M hydrochloric acid (HCl, Merck Titripur). The molar ratio of water to TEOS, referred to as the R-value, was 5. A 0.1 M HCl stock was used to adjust the pH of the final mixture to a value of 2. The hydrolysis reaction was allowed to proceed at room temperature (21-23° C.) for 25 minutes under continuous stirring. The silica sol was diluted with Milli-Q H₂O to an R-value of 55-56, and the pH was adjusted to 3.0±0.1 using 0.1 M sodium hydroxide (NaOH, Merck Titripur) solution. Next, 33.58 ml of the avacincaptad pegol-water solution was added to the silica sol and mixed. Last, the pH of the silica sol containing the avacincaptad pegol was adjusted to 6.0±0.1 with 0.1 M NaOH solution. The R-value of the final silica sol with soluble avacincaptad pegol was 100 with respect to components other than 0.1 M NaOH.

The sol comprising silica and ACP was pumped to the spray drier (SD, Buchi B-290) with an outlet temperature of between 52-66° C. to provide spray dried silica microparticles containing avacincaptad pegol. The ACP-silica microparticle are stored at 4-8° C. until further processing.

The described semi-batch process was used to prepare formulations varying the API drug loading, the first or primary R-value, and batch size as outlined in Table 1.

TABLE 1A
Feasibility formulations (microparticle) #01-#11

| | | Batch | | | | | | |
|---|---|---|---|---|---|---|---|---|
| | | size | 1^{st} | 2^{nd} | | | | |
| Formulation | API | (TEOS, | R- | R- | | | Inlet/Outlet | Effect |
| code | load¹ | ml) | value | value | Diluent | pH² | temperature³ | investigated |
| #01 | 10% | 2 | 5 | 100 | H₂O | 6 | 100° C./50° C. | API load |
| #02 | 20% | 2 | 5 | 100 | H₂O | 6 | 100° C./51° C. | API load |
| #03 | 40% | 2 | 5 | 100 | H₂O | 6 | 100° C./50° C. | API load |
| #04 | 20% | 2 | 10 | 100 | H₂O | 6 | 100° C./53° C. | 1st R-value |
| #05 | 20% | 2 | 15 | 100 | H₂O | 6 | 100° C./52° C. | 1st R-value |
| #06 | 20% | 2 | 20 | 100 | H₂O | 6 | 100° C./53° C. | 1st R-value |
| #07 | 25% | 2 | 5 | 100 | H₂O | 6 | 100° C./50° C. | API load |
| #08 | 30% | 2 | 5 | 100 | H₂O | 6 | 100° C./50° C. | API load |
| #09 | 35% | 2 | 5 | 100 | H₂O | 6 | 100° C./49° C. | API load |
| #02 (repeat) | 20% | 2 | 5 | 100 | H₂O | 6 | 100° C./51° C. | Batch size |
| #10 | 20% | 5 | 5 | 100 | H₂O | 6 | 100° C./51° C. | Batch size |
| #11 | 20% | 10 | 5 | 100 | H₂O | 6 | 100° C./51° C. | Batch size |

¹Target API load is defined as the ratio of theoretical API content in the microparticle to theoretical silica content, i.e., API-load = m_{API}/m_{SiO2}
²pH of the API-sol mixture at the onset of spray-drying
³Spray-drying inlet and outlet air temperatures

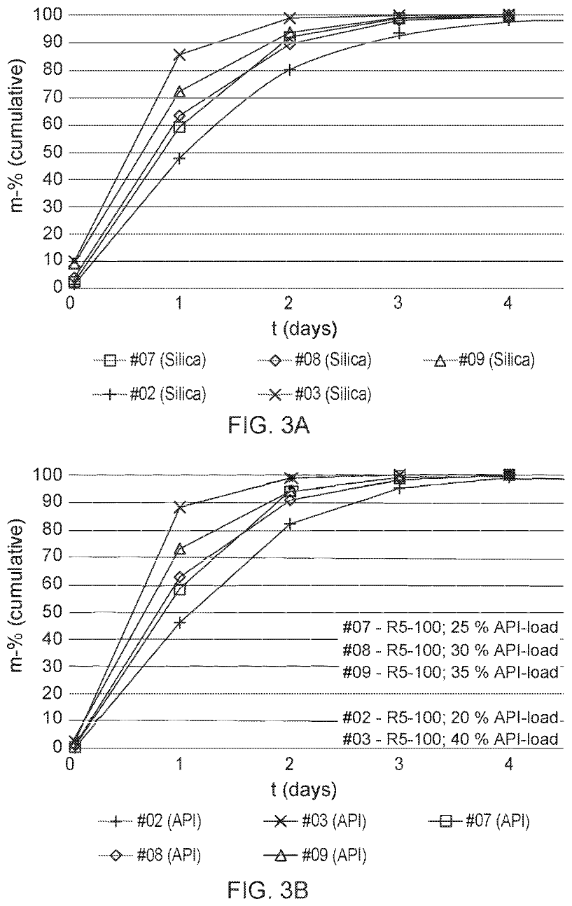

Feasibility formulations (microparticles) #01-#11 (Tables 1A and 1B) were characterized by light scattering particle size distribution, scanning electron microscopy, and in vitro dissolution. The results suggested that increasing API load accelerates in vitro dissolution (FIG. 3A, FIG. 3B). The first R-value had no measurable impact on light scattering particle size distribution or in vitro dissolution. The semi-batch process can be reproducibly prepared and batch size increased in the laboratory.

TABLE 1B
Feasibility formulations (microparticle) #01-#11 characterization

| | | Batch | | | | | | |
|---|---|---|---|---|---|---|---|---|
| | | size | | | | | API | |
| Formulation | API | (TEOS, | Silica | API | | Nonencaps | loading | Effect |
| code | load¹ | ml) | (%) | (mass %)² | Residuals | API (%) | (%)³ | investigated |
| #01 | 10% | 2 | 68.0 | 7.3 | 24.8 | <LLOQ | 10.7 | API load |
| #02 | 20% | 2 | 69.8 | 13.5 | 16.7 | 0.1 | 19.2 | API load |
| #03 | 40% | 2 | 61.0 | 24.0 | 15.0 | 1.5 | 36.9 | API load |
| #04 | 20% | 2 | 69.4 | 15.1 | 15.4 | 0.2 | 21.5 | 1st R-value |
| #05 | 20% | 2 | 69.0 | 14.9 | 15.8 | 0.1 | 21.3 | 1st R-value |
| #06 | 20% | 2 | 71.4 | 15.1 | 13.4 | 0.2 | 20.9 | 1st R-value |
| #07 | 25% | 2 | 66.6 | 23.1 | 10.0 | <LLOQ | 34.5 | API load |
| #08 | 30% | 2 | 63.7 | 22.3 | 13.4 | <LLOQ | 34.5 | API load |
| #09 | 35% | 2 | 61.4 | 27.0 | 10.7 | <LLOQ | 42.8 | API load |
| #02 (repeat) | 20% | 2 | 71.8 | 14.6 | 13.5 | <LLOQ | 20.3 | Batch size |
| #10 | 20% | 5 | 69.0 | 14.1 | 16.8 | <LLOQ | 20.4 | Batch size |
| #11 | 20% | 10 | 68.8 | 15.1 | 15.9 | <LLOQ | 21.9 | Batch size |

¹Target API load is defined as the ratio of theoretical API content in the microparticle to theoretical silica content, i.e., API-load = m_{API}/m_{SiO2}
²API % is mass fraction for total mass
³Measured API loading is defined as the ratio of measured API content in the microparticle to measured silica content, i.e., API-load = m_{API}/m_{SiO2}
LLOQ = lower limit of quantitation

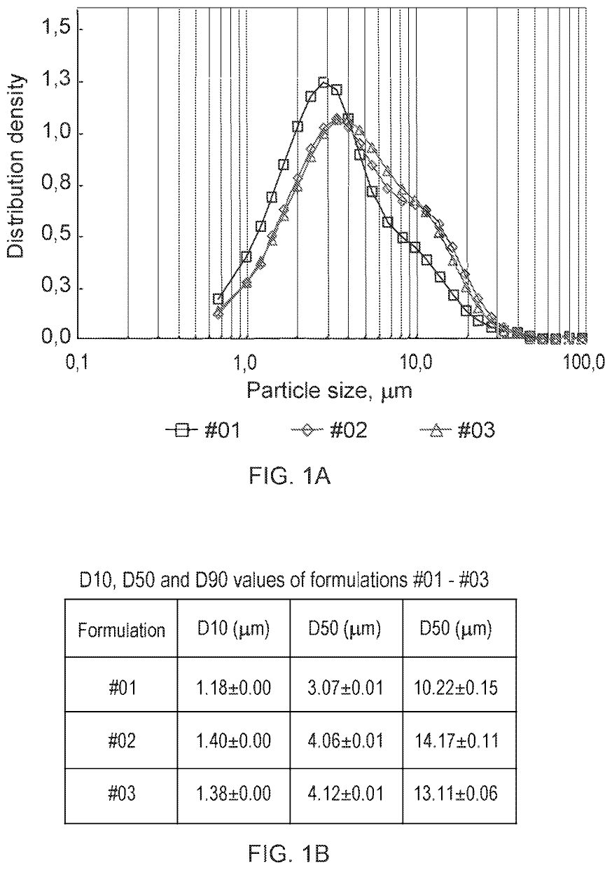

FIG. 1A shows the particle size distributions for exemplary microparticle formulations prepared by a semi-batch process as described in example 1A. Specifically, the figure shows the particle size distributions for microparticles #01-#03 in Table 1.

Further to Table 1, the target API load is defined as the ratio of theoretical API content (i.e., anti-C5 agent) in the microparticle to the theoretical silica content, i.e., API-load=m_{API}/m_{SiO2}. Further, the pH corresponds to the pH of the API-sol mixture at the onset of spray-drying. Further, the inlet/outlet temperature corresponds to the spray-drying inlet and outlet air temperatures.

In this regard, as depicted in FIG. 1A, the population of #01 is slightly separated from #02 and #03. Further, all of the formulations show a shoulder to the right of the distribution, which may indicate presence of particle aggregates and/or agglomerates.

FIG. 1B shows the D10, D50 and D90 values of formulations #01-#03. In this regard, the particle size distribution is appropriate for injection through narrow gauge needle. For example, the particle size values for D10, D50 and D90 can be at one of at 90%, 80%, 70%, 60%, 50%, 40%, 30%, 20%, or 10% of the needle internal diameter or lumen size.

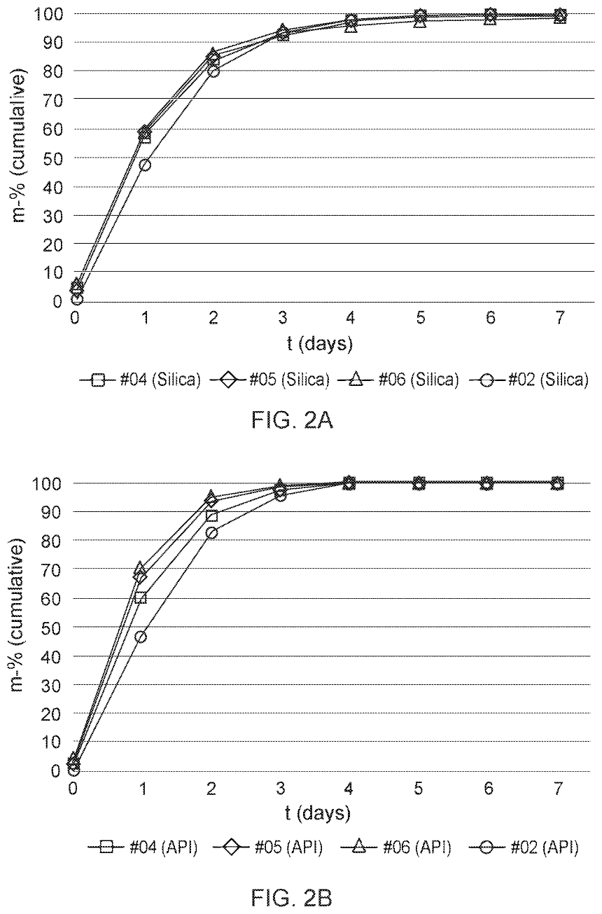

Particle size distribution for feasibility formulations #04-#06 is shown in Table 2. The microparticle formulations seem very similar. All formulations showed a shoulder to the right of the distribution, which may indicate the presence of particle agglomerates (microparticles that have fused together during spray drying). In vitro dissolution data for feasibility formulations #04-#06 is shown in FIG. 2A and FIG. 2B. Conclusion from formulations #04-#06 investigation is that 1^{st} R-value has no impact on in vitro dissolution or spray dried microparticle size distribution.

TABLE 2
D10, D50 and D90 values of formulations #04-#06

| Formulation | D10 (μm) | D50 (μm) | D90 (μm) |
|---|---|---|---|
| #04 | 1.50 ± 0.01 | 4.98 ± 0.15 | 14.89 ± 0.36 |
| #05 | 1.54 ± 0.2 | 5.32 ± 0.17 | 16.55 ± 0.34 |
| #06 | 1.54 ± 0.02 | 5.20 ± 0.21 | 16.08 ± 0.59 |

Drug loading evaluation 2: A second investigation of drug loading impact was performed to add granularity or fill in the gap between 20% and 40% loading investigated in formulations #02 and #03. Here, drug loading of 25%, 30%, and 35% were investigated. The data shows formulations #07-#09 have release rates that fall between the boundary formulations #02 and #03 with drug loading of 20% and 40%, respectively. The results support the conclusion that increased drug loading results in faster in vitro dissolution kinetics but does not impact particle size distribution of spray dried material.

Particle size distribution for feasibility formulations #07-#11 is shown in Table 3. All formulations showed a shoulder to the right of the distribution, which may indicate the presence of particle agglomerates. In vitro dissolution data for feasibility formulations #07-#09 is shown in FIG. 3A and FIG. 3B.

TABLE 3
D10, D50 and D90 values of formulations #07-#11

| Formulation | D10 (μm) | D50 (μm) | D90 (μm) |
|---|---|---|---|
| #07 | 1.19 ± 0.00 | 3.76 ± 0.02 | 11.48 ± 0.02 |
| #08 | 1.22 ± 0.00 | 3.79 ± 0.02 | 11.74 ± 0.08 |
| #09 | 1.28 ± 0.00 | 4.20 ± 0.03 | 12.52 ± 0.11 |
| #02 (repeat) | 1.06 ± 0.00 | 3.29 ± 0.01 | 10.46 ± 0.01 |
| #10 | 1.20 ± 0.00 | 3.40 ± 0.01 | 10.49 ± 0.11 |
| #11 | 1.19 ± 0.00 | 3.30 ± 0.00 | 11.48 ± 0.21 |

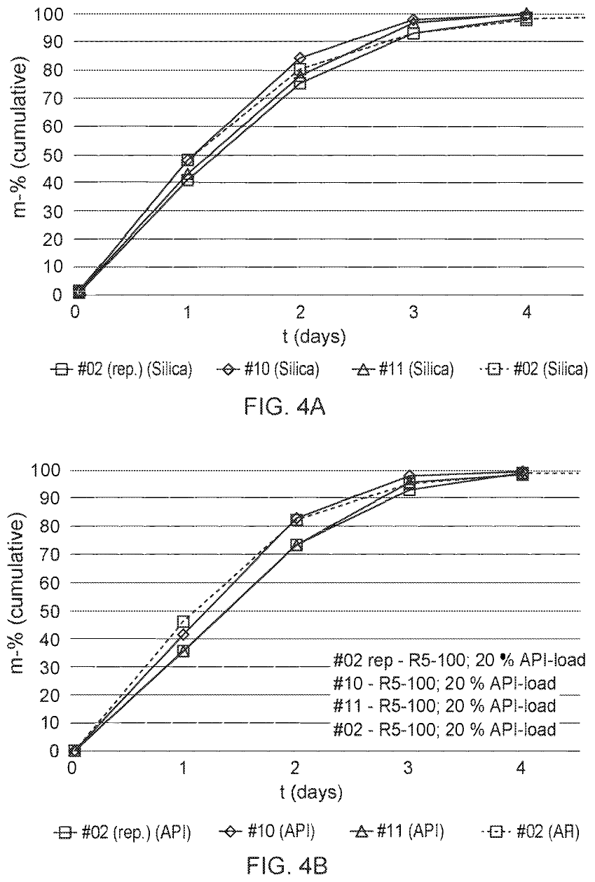

Scale up. The bench scale process was scaled based on input amount of silica source material (TEOS). In vitro dissolution data for feasibility formulations #02 (repeat), #10, and #11 is shown in FIG. 4A and FIG. 4B. The bench scale up work demonstrated that the silica input can be increased 5× without impacting the in vitro dissolution kinetics or particle size distribution of spray dried material.

### Example 1B: Preparation of Additional Exemplary Formulations Comprising an Anti-C5 Agent

Additional exemplary formulations as provided in Table 4 were generated and tested as described in more detail in the following Examples. Formulation Aging Study 1 was prepared using an appropriately scaled semi-batch process described in Example 1A. The remaining formulations annotated with CSTR (continuously stirred reactor) moniker were prepared according to example 2 scaled to the listed batch size. Formulation #11 CSTR-3 (PK) is also referred to as “PK” or “PK formulation” in subsequent Examples.

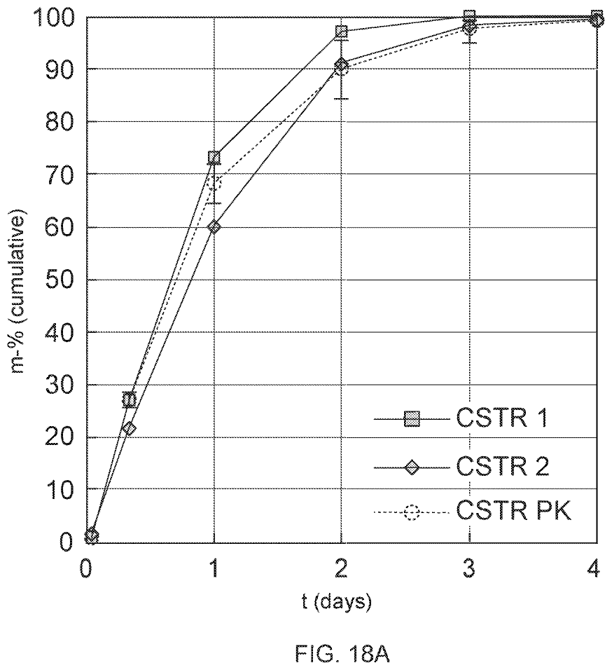

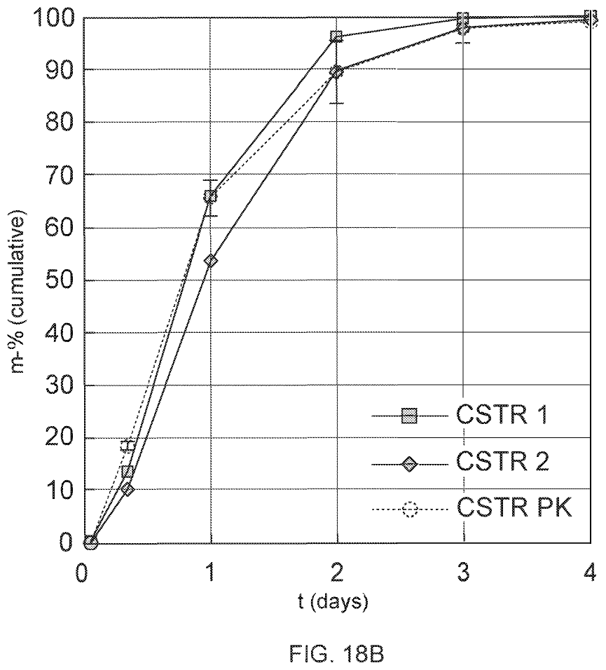

FIG. 18A and FIG. 18B show that silica and API dissolution rates are comparable for the #11 CSTR-1, #11 CSTR-2, and #11 CSTR-3 formulations. These data suggest that the CSTR batches have comparable dissolution release profiles.

TABLE 4A
Additional formulations (microparticles)

| | | Batch | | | | | | |
|---|---|---|---|---|---|---|---|---|
| | | size | 1^{st} | 2^{nd} | | | | |
| Formulation | API | (TEOS, | R- | R- | | | Inlet/Outlet | Effect |
| code | load¹ | ml) | value | value | Diluent | pH² | temperature³ | investigated |
| #11 CSTR-1 | 20% | 10 | 5 | 100 | H₂O | 6.0 | 100° C./52° C. | CSRT process |
| #11 CSTR-2 | 20% | 20 | 5 | 100 | H₂O | 6.2 | 100° C./62-66° C. | Batch size |
| #11 CSTR-3 (PK) | 20% | 70 | 5 | 100 | H₂O | 6.2-6.4 | 100° C./60-62° C. | Batch size |
| Aging Study 1 | 20% | 2 × 10 | 5 | 100 | H₂O | 6.0 | 100° C./50° C. | Batch process |
| (batch) | | | | | | | | |
| Aging Study 2 | 20% | 157.5 | 5 | 100 | H₂O | 6.0 | 100° C./52° C. | CSTR Process |
| (CSTR) | | | | | | | | |

¹Target API load is defined as the ratio of theoretical API content in the microparticle to theoretical silica content, i.e., API-load = m_{API}/m_{SiO2}
²pH of the API-sol mixture at the onset of spray-drying
³Spray-drying inlet and outlet air temperatures

TABLE 4b
Additional formulations (microparticles) characterization

| | | Batch | | | | | | |
|---|---|---|---|---|---|---|---|---|
| | | size | | | | | API | |
| Formulation | API | (TEOS, | Silica | API | | Nonencaps | loading | Effect |
| code | load¹ | ml) | (%) | (mass %)² | Residuals | API (%) | (%)³ | investigated |
| #11 CSTR-1 | 20% | 10 | 68.8 | 15.1 | 16.0 | <LLOQ | 21.9 | CSRT process |
| #11 CSTR-2 | 20% | 20 | 69.4 | 15.2 | 15.3 | <LLOQ | 21.8 | Batch size |
| #11 CSTR-3 (PK) | 20% | 70 | 65.8 | 17.7 | 16.4 | <LLOQ | 26.9 | Batch size |
| Aging Study 1 | 20% | 2 × 10 | 69.2 | 14.9 | 15.8 | <LLOQ | 21.6 | Batch process |
| (batch) | | | | | | | | |
| Aging Study 2 | 20% | 157.5 | 70.1 | 16.1 | 13.7 | <LLOQ | 23.0 | CSTR Process |
| (CSTR) | | | | | | | | |

¹Target API load is defined as the ratio of theoretical API content in the microparticle to theoretical silica content, i.e., API-load = m_{API}/m_{SiO2}
²API % is mass fraction for total mass
³Measured API loading is defined as the ratio of measured API content in the microparticle to measured silica content, i.e., API-load = m_{API}/m_{SiO2}

### Example 2: Preparation of Silica Microparticles Comprising an Anti-C5 Agent Employing a Continuously Stirred Reactor Process

The manufacture of silica microparticles was prepared using the following general procedure: preparation of anti-C5 agent (avacincaptad pegol) and NaOH solutions, preparation of silica sol by TEOS hydrolysis in a batch reactor, mixing of the component solutions (avacincaptad pegol solution, NaOH solution and silica sol) in a continuously-stirred tank reactor, and spray drying.

To prepare an avacincaptad pegol-water solution, 3.92 g of avacincaptad pegol is weighed and dissolved in 261.3 ml of milli-Q water to obtain a 15 mg/ml solution.

To prepare a NaOH solution, 16.17 ml 0.1 M NaOH solution and 269.21 ml milli-Q water were mixed such that a desired dilution is reached, wherein the pH of the solution should be 11.7.

TEOS was hydrolyzed in a batch reactor. The preparation of silica microparticles with 17.7% (w/w) encapsulated avacincaptad pegol was begun with the manufacture of silica sol. The sol is prepared by mixing the silica precursor, tetraethyl orthosilicate (TEOS, Sigma Aldrich), with milli-Q water (Merck Millipore) and 0.1 molar hydrochloric acid (Merck Titripur). The molar ratio of water to TEOS, referred to as the R-value, was 5. 0.1 M HCl was used such that the pH of the final mixture is 2. The hydrolysis reaction was allowed to proceed at room temperature (21-23° C.) for 25 minutes under continuous stirring.

The prepared solutions were combined in a continuously stirred tank reactor. The prepared solutions (silica sol, avacincaptad pegol-water solution, and NaOH-water solution) were pumped via a peristaltic pump (inlet pump) and mixed in a continuously stirred tank reactor (CSTR) at target volumetric flow ratios 1:3.21:2.83, respectively. The target mean residence time of the CSTR is 3-5 minutes. The concentration of silica should be within 16.7 and 30.1 mg/ml and the solution pH should be between 5.6-6.4. After mixing, the solution was pumped via a second peristaltic pump (outlet pump) to the spray drier (SD, Buchi B-290). The space time for the flow reactor step between the CSTR and the SD should be 1 minute. Outlet temperature of the spray dried should be between 52-66° C.

The following spray drying parameters were used: Aspirator (air): 538.3 L/min; Atomization air flow: 11.2 L/min; Nozzle type: Co-current two-fluid; Nozzle insert diameter: 0.7 mm; Nozzle cap diameter: 1.4 mm; Volumetric flow rate: 5.5-5.7 ml/min

The following represents total content assay results from microparticle formulation over a 1-week holding time:

TABLE 5

| | | Anti-C5 Agent | | |
|---|---|---|---|---|
| | | (avacincaptad | | API-loading |
| Time point | Silica (%) | pegol) (%) | Residuals (%) | (%) |
| 0 days | 65.8 ± 0.0 | 17.7 ± 0.4 | 16.4 ± 0.9 | 26.9 ± 1.1 |
| 7 days | 66.8 ± 1.0 | 17.7 ± 0.2 | 15.5 ± 1.1 | 26.4 ± 0.5 |

Total content assay of microparticles indicates that no material changes (e.g., no changes greater than experimental variability/measurement accuracy) have occurred during the 1-week holding time.

### Example 3: Preparation of a Silica Hydrogel Composite Comprising an Anti-C5 Agent

A silica hydrogel composite comprising an anti-C5 agent was prepared using the following general procedure. The avacincaptad pegol-silica microparticles from Example 2 were mixed with a dilute silica sol (separately prepared by TEOS hydrolysis) at a desired pH to obtain an avacincaptad pegol-silica-microparticle-silica sol suspension. The resultant suspension was then transferred to pre-filled syringes for primary packaging.

A silica sol with R-value of 400 was manufactured as described above. The pH of the silica sol was adjusted to 3 with 0.5 M NaOH. Next, silica microparticles with 17.7 weight-% encapsulated avacincaptad pegol were mixed with silica sol to reach 42.7 weight-% suspension. This step was performed under high shear mixing to minimize the presence of microparticle aggregates. After mixing, the microparticle suspension was transferred to the pre-filled syringes by injection via a needle from a reservoir syringe. Finally, the filled syringes were placed under vertical rotation for 6 days at room temperature.

A representative formulation for a hydrogel composition according to the present disclosure is shown in Table 6.

TABLE 6

| | Anti-C5 Agent | |
|---|---|---|
| Silica (%) | (avacincaptad pegol) (%) | Residuals (%) |
| 27.4 ± 2.9 | 8.0 ± 0.6 | 64.7 ± 3.5 |

In this regard, the anti-C5 agent payload is 4.6±0.3 (mg/50 μl). Further, the oligo-equivalent is 1.1±0.0 (mg/50 μl), wherein a factor of 0.23 was used for conversion. Further, the residuals account for mostly water and trace amounts of ethanol from TEOS hydrolysis, where n=3 for PK.

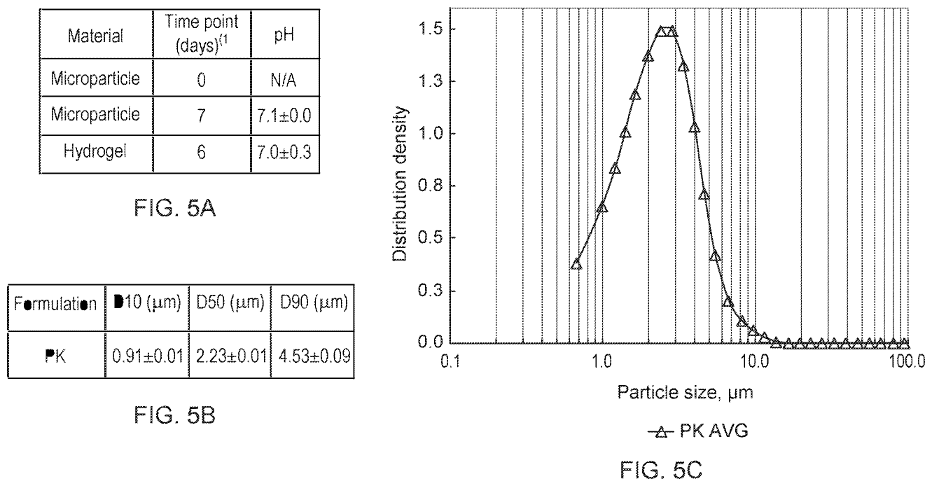

FIG. 5A shows the pH measurements of the microparticle and hydrogel formulations in Tables 5 and 6. In this regard, pH measurements of the microparticles are used as an aid to predict the resultant pH of the hydrogel, which has an impact on gelation kinetics (generally, the higher the pH, the faster the gelation process is).

FIG. 5B shows the D10, D50 and D90 values of the microparticle formulation in Table 5. FIG. 5C shows the particle size distribution of the microparticle formulation in Table 5. In this regard, as depicted in FIG. 5C, particle size distribution is very narrow and vast majority of the microparticles are below 10 μm, predicting adequate injectability.

### Example 4: SEM Morphology of Microparticles

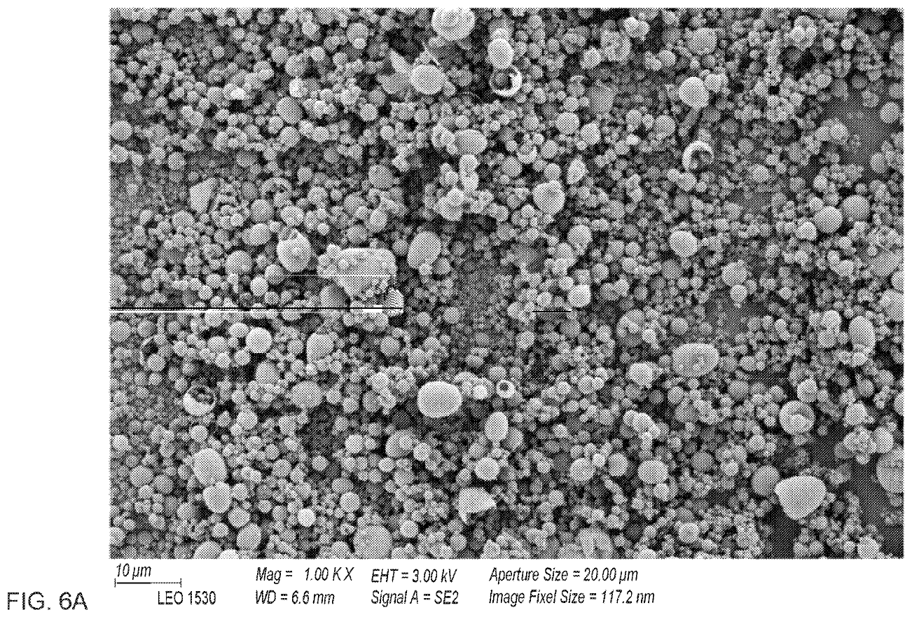

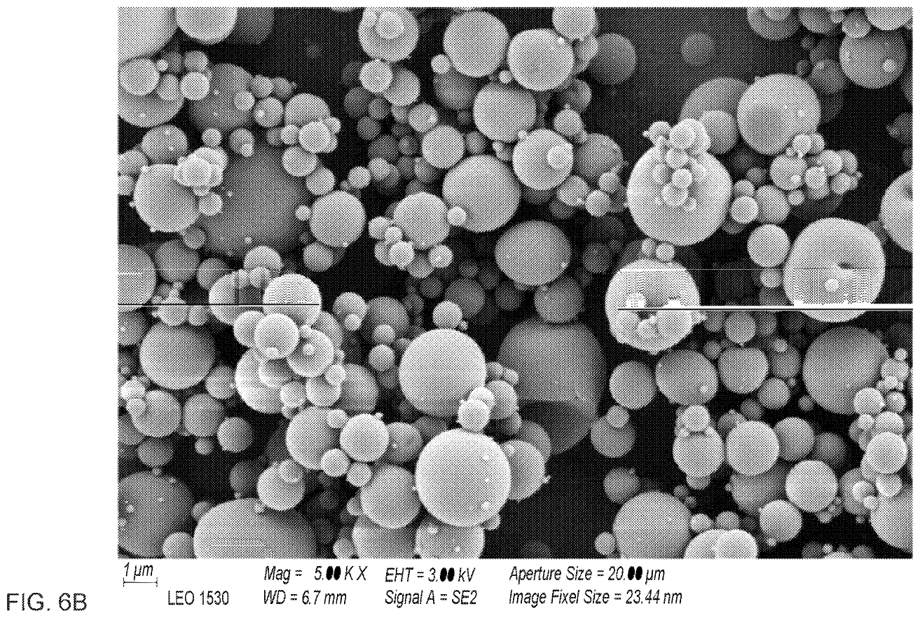

Particles are primarily spherical in nature and visually consistent in size with static light scattering size measurements. FIG. 6A shows the SEM imaging of the microparticle formulations in Table 5 at a first image magnification. In particular, the figure depicts the SEM imaging at the following parameters: mag=1.00 KX; EHT=3.00 kV; aperture size=20.00 μm; WD=6.6 mm; Signal A=SE2; image pixel size=117.2 nm. FIG. 6B shows the SEM imaging of the microparticle formulations in Table 5 at a second image magnification. In particular, the figure depicts the SEM imaging at the following parameters: mag=5.00 KX; EHT=3.00 kV; aperture size=20.00 μm; WD=6.7 mm; Signal A=SE2; image pixel size=23.44 nm. In this regard, as depicted in FIG. 6A and FIG. 6B, the particles appear smooth and spherical. Further, visually, the microparticle size appear to agree with the particle size distribution measurement, e.g., vast majority of the microparticles are less than 5 μm in diameter. Lastly, no discernable presence of microparticle agglomerates can be seen, as suggested by the particle size distribution profile in FIG. 5C.

### Example 5: In Vitro Release of Anti-C5 Agent from Silica Composite

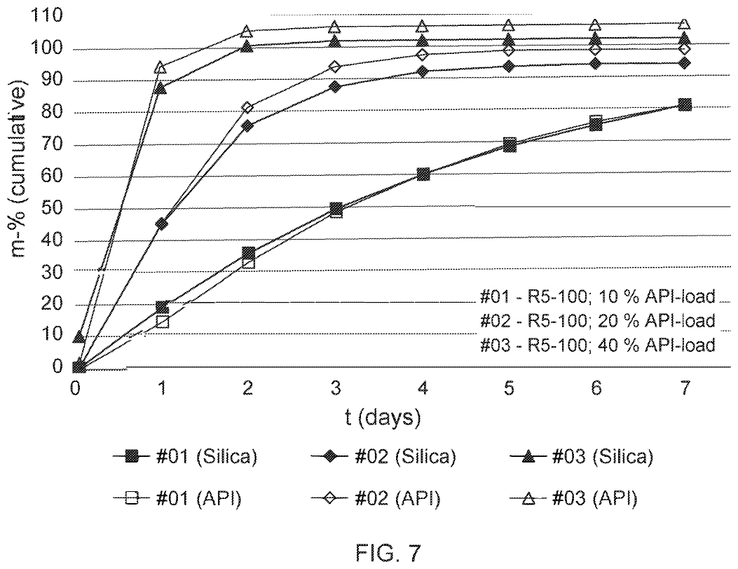

FIG. 7 shows the in vitro release of avacincaptad pegol and silica matrix degradation in the microparticles #01-#03 in Table 1. In this regard, as depicted in the figure, API release rates are affected by the API load in the microparticle. Further, the API is effectively encapsulated by the silica matrix as indicated by very low release at 1^{st} hour timepoint. Further, the release of the API is controlled by the degradation of the silica matrix.

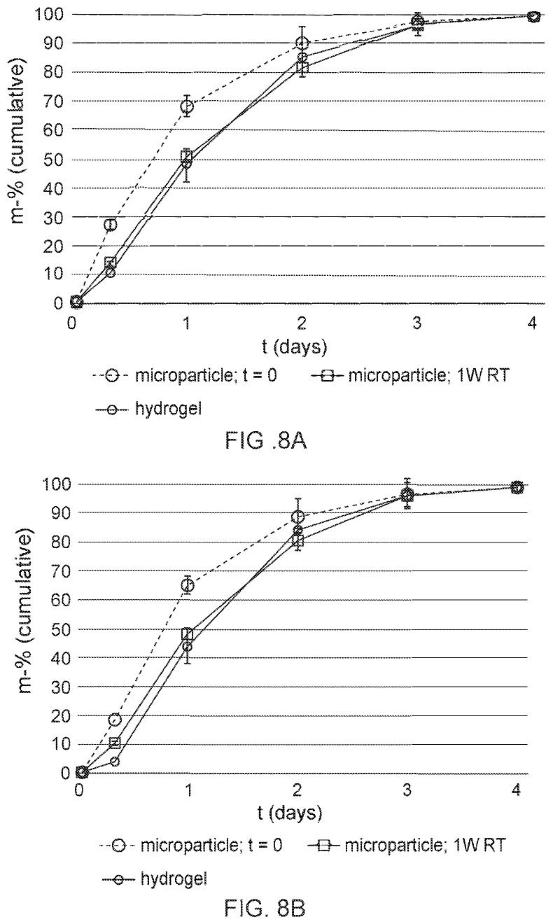

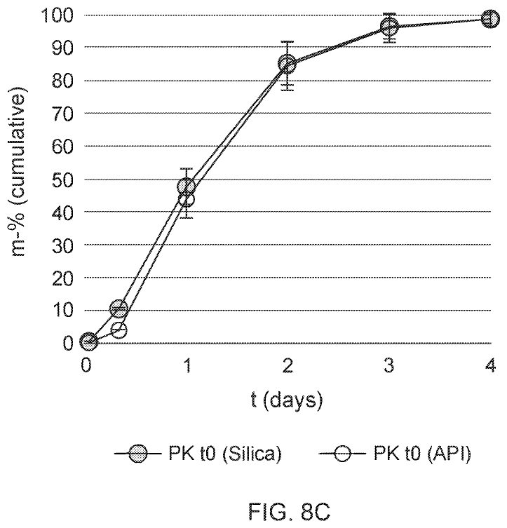

FIG. 8A shows the silica matrix degradation of the microparticle and hydrogel formulations in Tables 5 and 6. FIG. 8B shows the API release of the microparticle and hydrogel formulations in Tables 5 and 6. In this regard, the API release and silica matrix degradation of FIG. 8A and FIG. 8B occurred at 50 ml dissolution volume. Further, the microparticles were kept at ambient temperature for one week prior to the composite hydrogel manufacture, the purpose of which was to deliberately alter the silica matrix degradation and API release kinetics. As depicted in FIG. 8A and FIG. 8B, the microparticles appear to retain the silica matrix degradation and API release rates, respectively. Further, the API release rate is not impacted by preparing the silica composite relative to the silica microparticles.

FIG. 8C shows the direct relationship between silica and API dissolution. This is a surprising advantage over other delivery technologies where the matrix that controls drug release can last several times longer than API release leading to build-up or residual matrix in the eye over time.

### Example 6: In Vivo Ocular Release of Anti-C5 Agent from Silica Composite

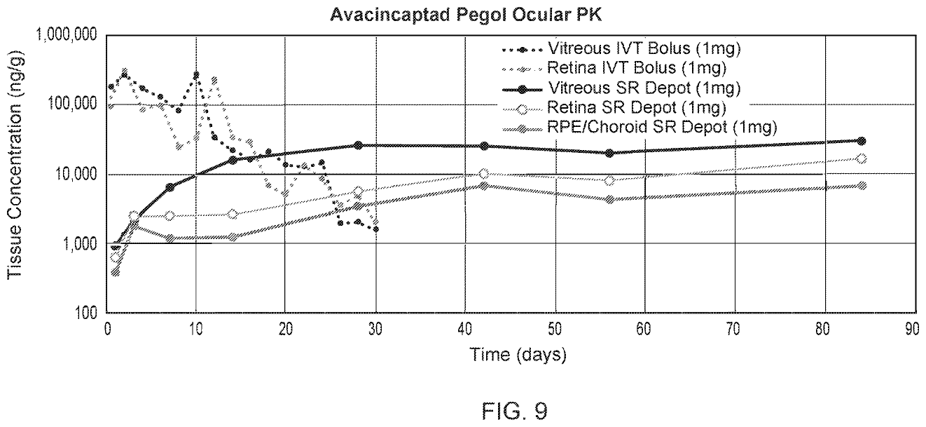

The composite formulation (using formulation #11 CSTR-3) prepared as described in Examples 2 and 3, whose in vitro release profile is shown in FIG. 8A and FIG. 8B, was evaluated for ACP release and tissue exposure following IVT administration in the Dutch belted (DB) rabbit. The study design includes bilateral intravitreal dosing of 1.0 mg oligo equivalent in a 50 μL dosing volume of silica composite PK formulation. Two rabbits (4 eyes) were collected at each of the following time points, day 1, 3, 7, 14, 28, 42, 56, and 84. Eyes were dissected, and vitreous humor, retina, and retinal pigment epithelium (RPE)/choroid were collected for tissue specific bioanalytical analysis employing a qualified dual-hybridization assay. The ocular PK (pharmacokinetics) is summarized in FIG. 9 for liquid formulation administered as an IVT bolus dose for vitreous humor and retina tissues as comparator to the sustained release depot PK formulation. The ocular tissue PK for the PK formulation is provided as solid lines for vitreous humor (top line), retina (middle line) and RPE/choroid (bottom line). The data demonstrate that a single dose administration of the sustained release PK formulation of Example 3 provides prolonged and consistent ocular tissue concentrations for up to 84 days.

At study termination (day 84), the remaining PK formulation depot was recovered from the vitreous humor by centrifugation and analyzed for remaining silica by microwave plasma atomic emission spectroscopy (MP-AES) method and avacincaptad pegol content by a qualified HPLC assay method. The remaining silicon ranged between 0-9% and avacincaptad pegol ranged from 0-3% of the administered dose.

The in vitro dissolution data provides an estimated duration of release based on a historic in vitro-in vivo correlation (IVIVC) of 30×. Based on FIG. 8B the predicted duration to ˜95% release is 3×30=90 days. This was used to predict an ACP release rate from the depot formulation (1.0 mg/90 days=11 micrograms per day. The terminal half-life of ACP in DB-rabbits following bolus administration of a liquid formulation was used to predict the steady state vitreous humor concentration based on a daily input rate of 11 micrograms per day and terminal half-life of 3.8 days. This calculation estimated a vitreous humor steady state concentration of ˜30,000 ng/mL. The average measured steady state vitreous humor concentration was from day 28 to day 84 was 22,650 ng/g. For this analysis the density of vitreous humor is approximated as 1 so the measured steady state is 75% of the predicted value. Further, analysis of the depot formulation remnant recovered at day 84 indicated <10% formulation remaining (range 0-9%) which supports approximately 3 month or 90 day predicted duration.

### Example 7: Stability Assessment

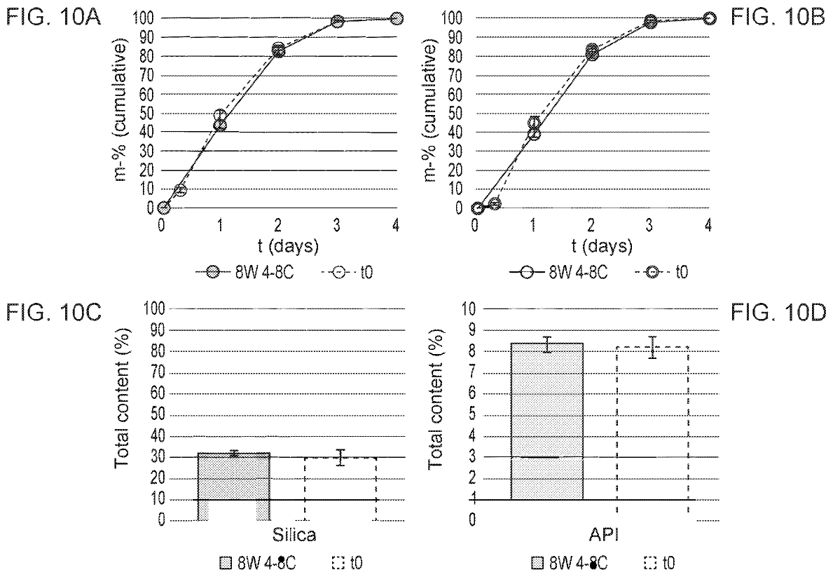

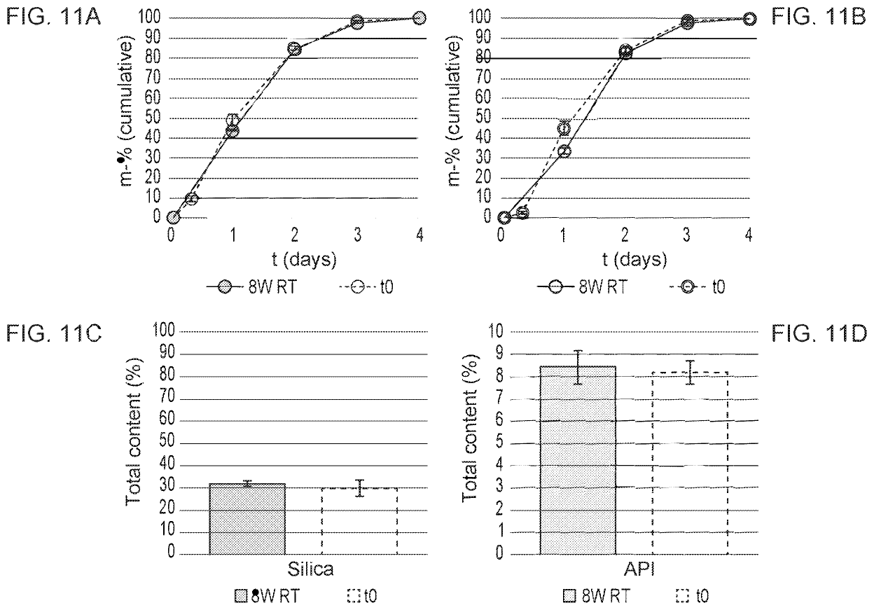

PK formulation of Example 3 was packaged in a 0.5 mL RTF® glass LuerLock syringe with a Datwyler Neoflex stopper and evaluated for stability under refrigerated (2-8° C.) and room temperature storage condition for 8 weeks. Results for stability at 2-8° C. are shown in FIG. 10A-FIG. 10D. Results for stability at room temperature are shown in FIG. 11A-FIG. 11D. The stability data at both storage conditions highlights there is no change in PK formulation silica content, API content, or in vitro release kinetics at either storage condition.

### Example 8: Aging Study

An interesting observation was made during storage of ACP microparticles compared to the stability of the composite depot formulation of Example 7. A measurable decrease is release rate was observed as a function of storage time and temperature.

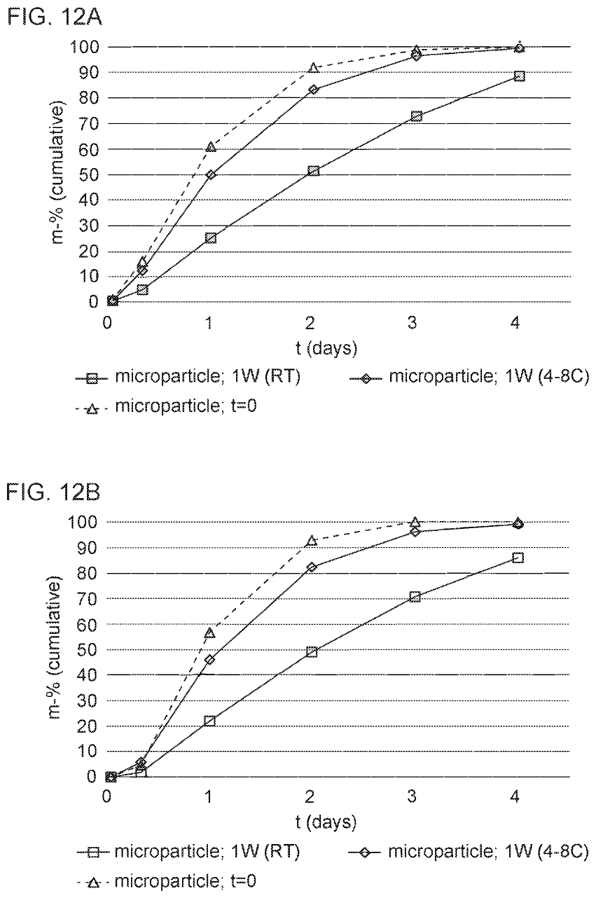

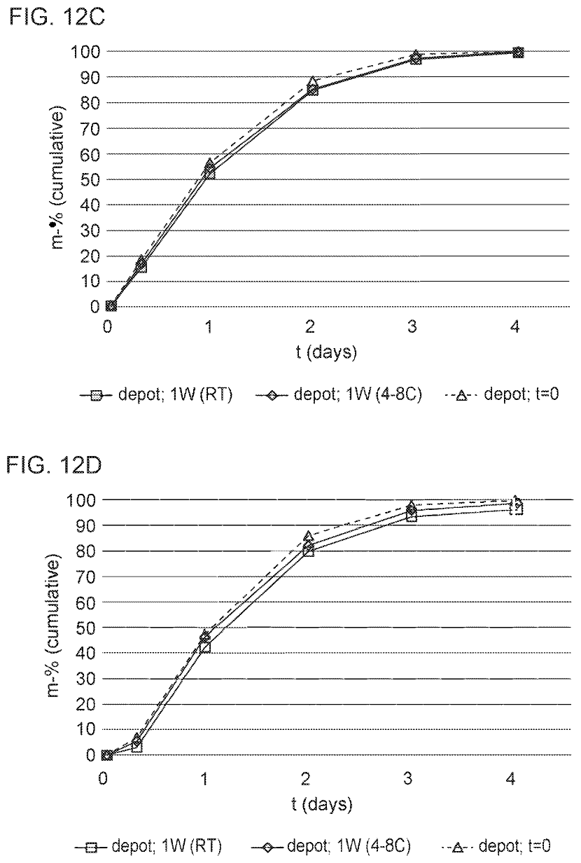

FIG. 12A-FIG. 12D illustrate the change in dissolution rate of silica (FIG. 12A) and ACP (upper right panel) from silica microparticles following one week at refrigerated and room temperature storage conditions. In contrast the dissolution rate of silica (FIG. 12C) and ACP (FIG. 12D) from the silica depot composite are unchanged following one week at refrigerated and room temperature storage conditions.

Aging was investigated further to assess the impact of alternative storage conditions including vacuum, nitrogen atmosphere, and high humidity. ACP silica based microparticles were prepared according to the method of Example 1B as a scale to provide 38 grams of ACP-microparticles. This provided microparticles with a similar particle size distribution as the PK formulation of Example 2 and a slightly narrower size distribution than the microparticles of the first aging study.

TABLE 7
Particle size distribution of PK, Aging study
1 and aging study 2 ACP microparticles

| Material | D10 (μm) | D50 (μm) | D90 (μm) |
|---|---|---|---|
| PK formulation | 0.91 ± 0.01 | 2.23 ± 0.01 | 4.53 ± 0.09 |
| Aging study 1 (batch) | 1.19 ± 0.00 | 3.30 ± 0.00 | 11.48 ± 0.21 |
| Aging study 2 (CSTR) | 1.44 ± 0.00 | 2.75 ± 0.00 | 4.96 ± 0.01 |

ACP-microparticle were aliquoted into glass vials, and loosely capped to allow full exposure to the environmental storage condition. Storage was performed at room temperature with different atmospheric conditions including vacuum, nitrogen, and greater than 95% relative humidity. Under all storage conditions the direct relationship of silica to ACP dissolution was maintained, and the dissolution rates decreased as a function of time. The largest change in dissolution rate is for the nitrogen storage condition, and the smallest change is for the greater than 95% relative humidity sample. The data demonstrates that atmospheric storage conditions impact aging in addition to temperate demonstrated in the first aging study. Aging provides a tool that can be exploited to tune the dissolution kinetics of ACP based silica microparticles. Note that the stability data of Example 7 demonstrates the aging phenomenon does not occur in the final ACP composite depot formulation of ACP-microparticles suspended in a silica-sol hydrogel.

### Example 9: Composite Depot Rheology

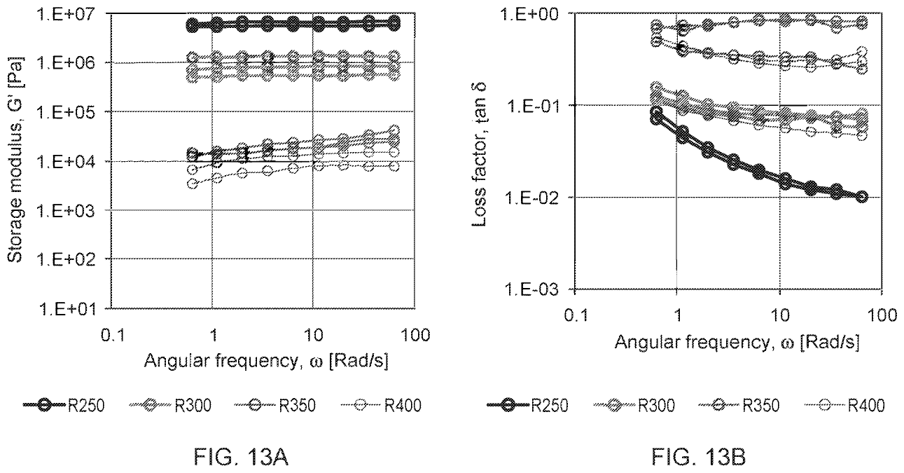

In the present disclosure, silica can be used to create a silica sol hydrogel depot, thus obviating the need for new or additional materials or excipients in the composite depot formulation that could complicate biocompatibility. The rheology of the final composite depot is a property that may impact the depot stability, injectability, and performance post dose administration. The silica sol hydrogel creates a depot formulation of silica based microparticles dispersed within a silica sol hydrogel. Microparticles were prepared according to the procedure of Example 1A using the composition of formulation #11 at an input scale of 40 mL TEOS. Four separate composite depots were prepared using the microparticle according to the method of Example 2 with hydrogel R-vales of 400, 350, 300, and 250 respectively. Results are shown in FIG. 13A and FIG. 13B.

The rheology of the prepared composite depot was assessed. Rheological measurements of API-silica microparticle-silica hydrogel composite materials were carried out using an Anton Paar MCR 302 (Modular Compact Rheometer). A decreasing hydrogel R-value correlates with an increase in storage modulus (G′) and a decrease in the loss factor (tan δ) of the composite depot. The results indicated that the composite depot rheology can be controlled by the silica content of the prepared silica-sol hydrogel and increased silica content improves the mechanical properties of the composite depot.

### Example 10: Formulation Development

Experiments were executed to probe the impact of changing the pH of the final silica-sol for composite depot production, 1^{st} R-value, 2^{nd} R-value, and reaction time. The batches were prepared using the continuous flow reactor process of Example 2 and composition as listed in Table 8.

TABLE 8
Formulation Development Parameters

| | | 1st | 2nd | | | Rxn | Volumetric | Particle | Outlet | Room | Relative |
|---|---|---|---|---|---|---|---|---|---|---|---|
| | | R | R | pH | | t | flow rate | collection | T | T | Humidity |
| # | Code | value | value | (target) | | (min) | (ml/min) | time (min) | (° C.) | (° C.) | (%) |
| ref. 1 | PK | 5 | 100 | 6.2-6.3 | (6) | 1 | 5.7 | ≈100 | 60-62 | 22.7- | 39-35 |
| | | | | | | | | | | 22.9 | (desc.) |
| ref. 2 | #11 | 5 | 100 | 6.0 | (6) | N/A | 5.7 | 19 min | 51 | 23.4 | 29 |
| 1 | pH #1 | 5 | 100 | 4.9-5.0 | (4) | 1 | 5.7 | 12-15 | 60 | 22.1 | 44 |
| 2 | pH #2 | 5 | 100 | 5.0-5.3 | (5) | 1 | 5.7 | 12-15 | 60 | 22.4 | 44 |
| 3 | pH #3 | 5 | 100 | 7.7-7.8 | (7) | 1 | 5.7 | 12-15 | 61 | 22.4 | 44 |
| 4 | 2R #1 | 5 | 150 | 6.1-6.2 | (6) | 1 | 5.7 | 14 + 10 | 59 | 23.1 | 39 |
| 5 | 2R #2 | 5 | 200 | 6.1-6.3 | (6) | 1 | 5.7 | 25 | 60 | 22.7 | 34 |
| 6 | 2R #3 | 5 | 250 | 6.0-6.2 | (6) | 1 | 5.7 | 25 | 59-60 | 22.7 | 34 |
| 7 | Rxn #1 | 5 | 100 | 6.2-6.3 | (6) | 10 | 5.0 | 12 | 64 | 23.9 | 51 |
| 8 | Rxn #2 | 5 | 100 | 6.3 | (6) | 8 | 5.0 | 12 | 62 | 22.5 | 52 |
| 9 | Rxn #3 | 5 | 100 | 6.2-6.3 | (6) | 5 | 5.0 | 12 | 63 | 24.1 | 51 |
| 10 | 1R #1 | 3 | 100 | 6.1-7.0 | (6) | 1 | 5.0 | 12 | 61 | 23.6 | 44 |
| 11 | 1R #2 | 10 | 100 | 6.2-6.3 | (6) | 1 | 5.0 | 12 | 62 | 22.3 | 46 |
| 12 | 1R #3 | 20 | 100 | 5.4-5.5 | (6) | 1 | 5.0 | 12 | 61 | 23.9 | 46 |

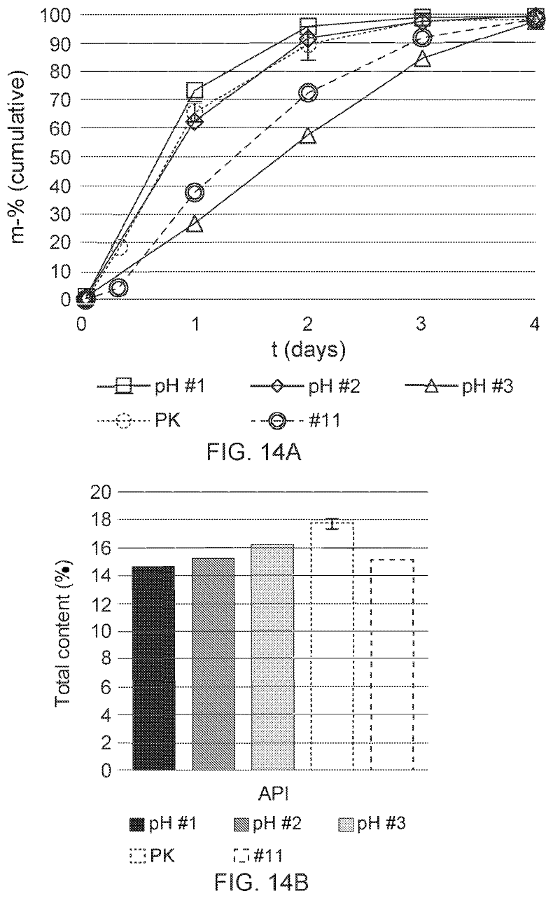

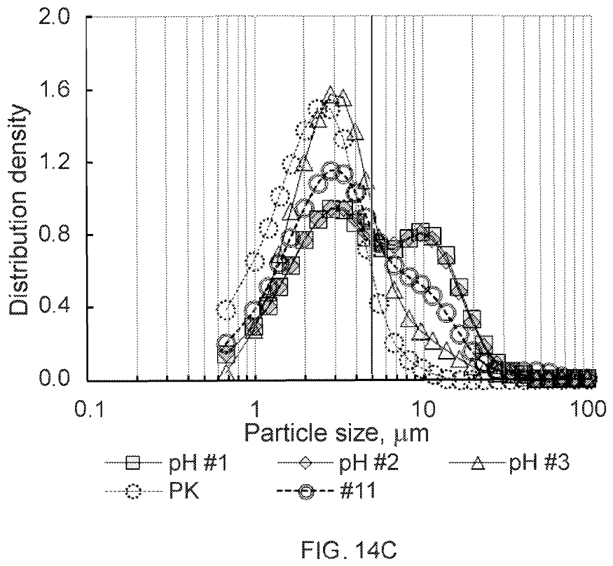

Formulations pH #1-pH #3 indicate that the dissolution profile has a trend of slower release at pH 7 versus pH 4.9-5.3 and microparticle size distribution. The pH 4.9-5.3 formulation had a bimodal microparticle size distribution indicating possible particle aggregation under these processing conditions. The pH range investigated had a minimal effect on silica or drug loading. Results are shown in FIG. 14A-FIG. 14C.

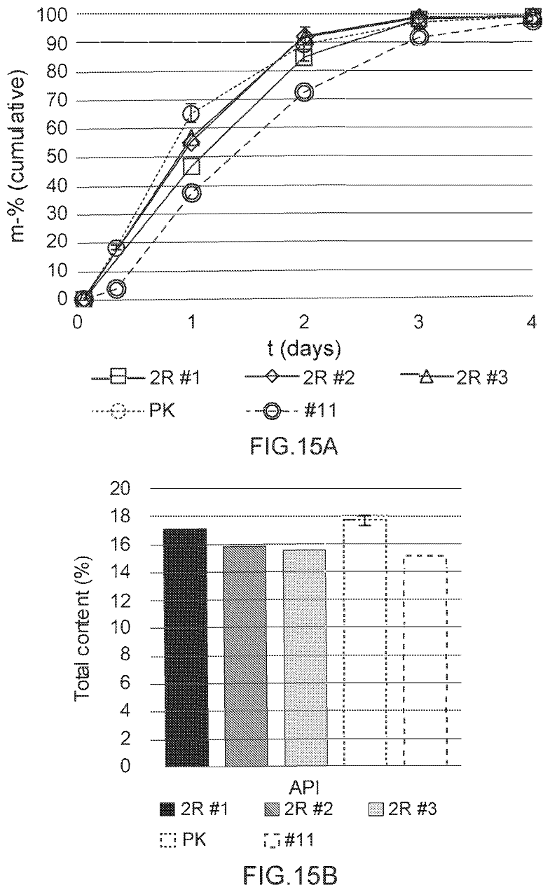

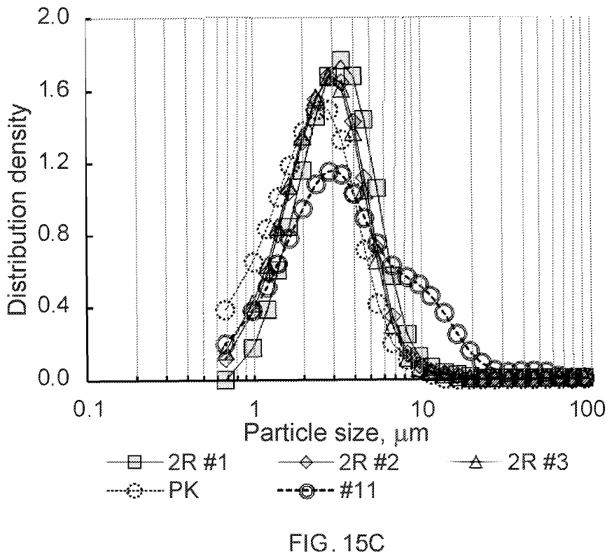

Formulations 2R #1-2R #3 reveal the secondary R-value has minimal effect on dissolution, microparticle size distribution, silica, and drug loading. Results are shown in FIG. 15A-FIG. 15C.

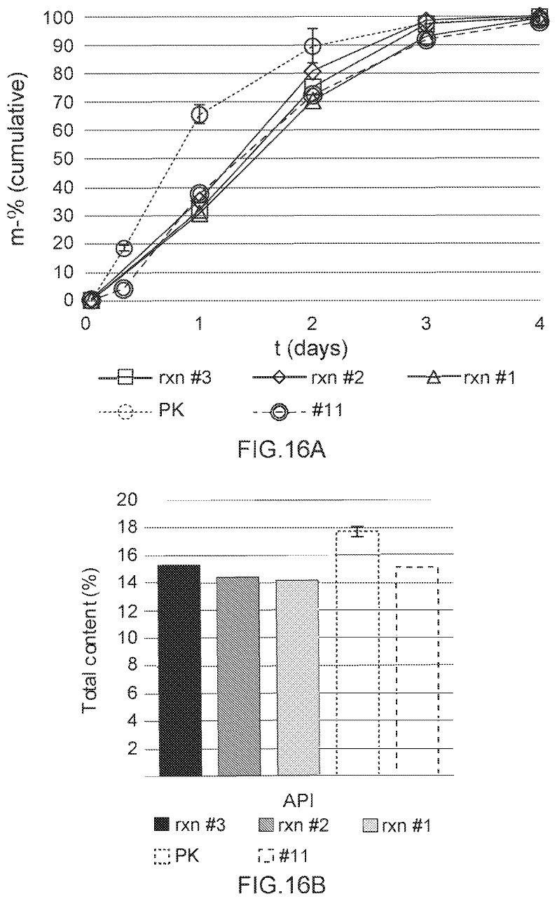

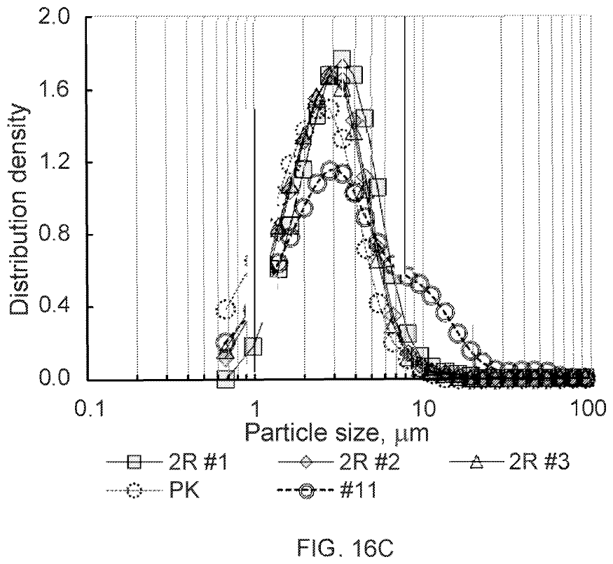

Formulations Rxn #1-Rxn #3 evaluated the impact of reaction time from 10 down to 5 minutes. There is a potential trend over the time range of slower dissolution rates as the reaction time increases. There was minimal impact on drug loading and no impact on microparticle size distribution. Results are shown in FIG. 16A-FIG. 16C.

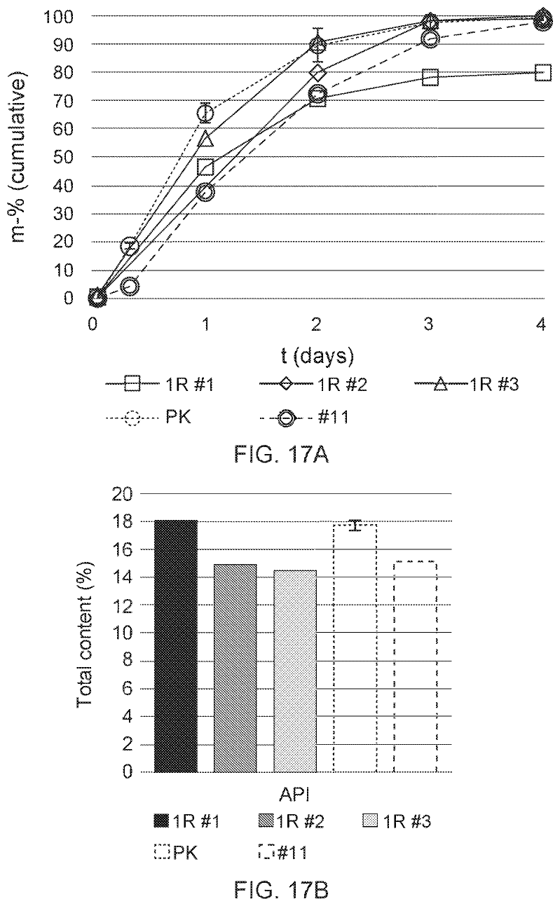

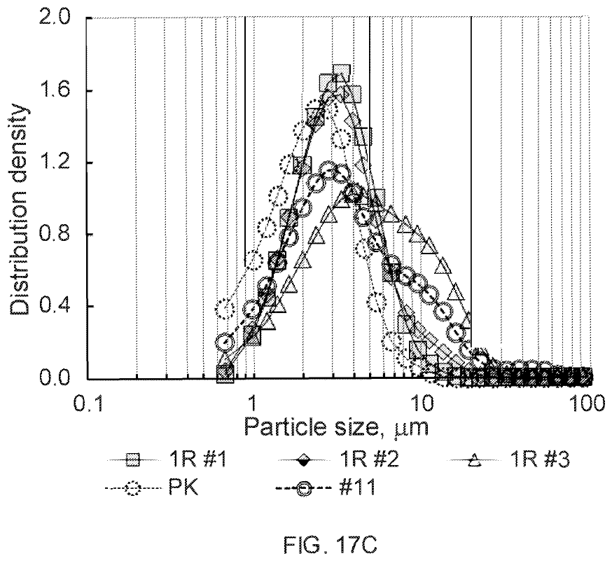

Formulations 1R #1-1R #3 probed the effect of the primary R-value to a lower value than evaluated previously. Precipitation was noted at the primary R-value of 3 which impacted the total amount of avacincaptad pegol released. Formulation 1R #3 was prepared at a lower than target pH value so two of the three formulations in this sequence were compromised limiting the conclusions that can be drawn from this subset of formulations. Results are shown in FIG. 17A-FIG. 17C.

### Example 11: Experimental Methods

The experimental methods for preceding Examples are briefly described as follows.

### In Vitro Degradation.

Microparticle samples: In vitro degradation of silica and the release of the API was measured in PBS (137 mM NaCl, 2.7 mM KCl, 10 mM Na₂HPO₄, 1.8 mM KH₂PO₄) supplemented with 0.05% TWEEN80 (pH 7.4 at +37° C.). The size of the microparticle or hydrogel sample analyzed was ca. 10-25 mg, and the dissolution studies were conducted up to 96 hours in a shaking water bath (60 strokes/min).

Hydrogel depot samples: The buffer and conditions for the hydrogel depot samples were identical to microparticle samples described in the above section. The amount of sample weighed for the analysis ranges from ca. 15-25 mg. In addition, the sample preparation was different, such that the hydrogel depot was dispersed into the dissolution buffer by placing a small magnet into the jar and mixing until the suspension appeared homogenous. This was done to ensure that the dissolution conditions of the hydrogel depots were comparable to microparticles, i.e., the shape of the hydrogel depot in the dissolution jar would not impact its degradation profile due to a difference in available surface area for dissolution.

### Silica- and ACP Total Content Assays

Silica concentrations were measured with Microwave Plasma Emission Spectroscopy (MP-AES) analyzing the electron emission intensity with wavelength of 261 nm. The API was analyzed with a high-performance liquid chromatograph (HPLC) connected to diode array detector (at λ=258 nm). The chromatographic separation was obtained on an DNAPac PA-100, 4×250 mm, 13 μm with Guard Column DNAPac PA-100, 4×50 mm, ThermoScientific.

### Particle Size Distribution (PSD)

The particle size distribution measurements of the API-silica microparticles were carried out by using a static light scattering method. The instrument used was a Sympatec HELOS BR3 using an R3 lens optimal for microparticle size range of 0.5 through 175 μm. The sample was prepared by weighing ca. 20 mg of API-silica microparticles and adding about 3 ml of Milli-Q H2O. Next, the suspension was vortex-mixed for 30 seconds with full power. Then, 60 μl of the suspension was pipetted into a cuvette (V≈35 ml) filled with Milli-Q H2O. The sample was sheared with ultrasound for 20 seconds prior to the measurement.

### Manual Injectability

The injectability of the silica-API microparticle-silica hydrogel composite formulations was tested by manually injecting the material. First, a 27G TUTW hypodermic needle was attached to the syringe. Next, the syringe was primed such that material filled the needle hub entirely and the needle was wiped clean of any excess material. Finally, the primed syringe was emptied onto a petri dish. The injection was considered successful if the motion was continuous, i.e., no transient blockages occurred during the procedure. The resultant material was also visually inspected.

The foregoing description is given for clearness of understanding only, and no unnecessary limitations should be understood therefrom, as modifications within the scope of the disclosure may be apparent to those having ordinary skill in the art.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The presently disclosed subject matter illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. In case of conflict between the present disclosure and incorporated references, articles, publications, patents, patent publications, and patent applications, the present disclosure should control.

1. A sustained release silica hydrogel composite, the composite comprising: silica content in the range of 5-35% and anti-C5 agent in the range of 1-40%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUAfCf CfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

2. The sustained release silica hydrogel composite of claim 1, wherein the composite comprises silica content in the range of 5-35% and anti-C5 agent in the range of 5-40%.

3. The sustained release silica hydrogel composite of claim 1, wherein the composite comprises silica content in the range of 5-30% and anti-C5 agent in the range of 1-5%, 5-10%, 10-15%, 15-20%, 20-25%, or 25-30%.

4. The sustained release silica hydrogel composite of claim 1, wherein the composite comprises silica content in the range of 25-30% and anti-C5 agent in the range of 5-10%.

5. The sustained release silica hydrogel composite of claim 1, wherein the composite comprises silica content of about 27.4% and anti-C5 agent of about 8%.

6. The sustained release silica hydrogel composite of claim 1, wherein the composite comprises silica microparticles dispersed in silica-sol hydrogel.

7. The sustained release silica hydrogel composite of claim 1, wherein the composite has a 2:1 ratio, 1:1 ratio, or 1:2 ratio of silica dissolution rate to anti-C5 agent dissolution rate.

8. The sustained release silica hydrogel composite of claim 1, wherein the anti-C5 agent is pegylated.

9. The sustained release silica hydrogel composite of claim 1, wherein the anti-C5 agent is unpegylated.

10. A syringe comprising the sustained release silica hydrogel composite of claim 1.

11. A method for ameliorating, treating or reducing the severity of a symptom of an ophthalmological condition in a subject in need thereof, the method comprising administering to the subject the sustained release silica hydrogel composite of claim 1.

12. A method for preventing or delaying the progression of an ophthalmological condition in a subject in need thereof, the method comprising administering to the subject the sustained release silica hydrogel composite of claim 1.

13. A method for treating or reducing the severity of an ophthalmological condition in a subject in need thereof, the method comprising administering to the subject the sustained release silica hydrogel composite of claim 1.

14. The method of claim 11, wherein the ophthalmological condition is incomplete retinal pigment epithelial (RPE) and outer retinal atrophy, complete RPE and outer retinal atrophy, nascent geographic atrophy, geographic atrophy, or wet age-related macular degeneration.

15. The method of claim 11, wherein the sustained release silica hydrogel composite is administered to the subject by subconjunctival, retrobulbar, intracameral, sub-tenon, sub-retinal, suprachoroidal, or intravitreal injection.

16. The method of claim 11, wherein the sustained release silica hydrogel composite is administered to the subject by intravitreal injection.

17. The method of claim 11, wherein the sustained release silica hydrogel composite is administered to the subject by suprachoroidal injection.

18. The method of claim 11, wherein the sustained release silica hydrogel composite is administered to the subject at a dose of from about 0.3 mg/eye to about 5 mg/eye.

19. The method of claim 11, wherein the sustained release silica hydrogel composite is administered to the subject at a dose of about 2 mg/eye.

20. The method of claim 11, wherein the sustained release silica hydrogel composite is administered to the subject at a frequency in which the duration between doses is at least about three months.

21. The method of claim 11, wherein the sustained release silica hydrogel composite is administered to the subject at a frequency in which the duration between doses is about four months, about five months, or about six months.

22. A formulation comprising a population of microparticles, the microparticles comprising: silica content in the range of 10-70% and anti-C5 agent in the range of 5-50%, wherein the anti-C5 agent comprises a C5-specific aptamer, in which the aptamer comprises a nucleotide sequence of fCmGfCfCGfCmGmGfUfCfUfCmAmGmGfCGfCfUmGmAmGfUfCfUmGmAmGfUfUfUAfCf CfUmGfCmG-3T (SEQ ID NO: 1), in which fC and fU=2′ fluoro nucleotides, mG and mA=2′-OMe nucleotides, all other nucleotides are 2′-OH, and 3T indicates an inverted deoxythymidine.

23. The formulation of claim 22, wherein the microparticles comprise silica content in the range of 60-75% and anti-C5 agent in the range of 2.5-5.0%, 5-10%, 10-15%, 15-20%, 20-25%, or 25-30%.

24. The formulation of claim 22, wherein the microparticles comprise silica content in the range of 60-72% and anti-C5 agent in the range of 2.5-25%.

25. The formulation of claim 22, wherein the microparticles comprise silica content in the range of 64-68% and anti-C5 agent in the range of 15-19%.

26. The formulation of claim 22, wherein the anti-C5 agent is pegylated.

27. The formulation of claim 22, wherein the anti-C5 agent is unpegylated.
